(19) United States
(12) Patent Application Publication
(10) Pub. No.: US 2014/0094610 A1
(43) Pub. Date: Apr. 3, 2014

(54) PROCESS FOR THE PREPARATION OF AN HIV INTEGRASE INHIBITOR

(71) Applicant: Gilead Sciences, Inc., Foster City, CA (US)

(72) Inventors: Wenjie LI, Hopewell Junction, NY (US); Philomen DECROOS, Middlebury, CT (US); Keith R. FANDRICK, Sandy Hook, CT (US); Joe Ju GAO, Southbury, CT (US); Nizar HADDAD, Danbury, CT (US); Zhi-Hui LU, Newtown, CT (US); Bo QU, Brookfield, CT (US); Sonia RODRIGUEZ, New Milford, CT (US); Chris H. SENANAYAKE, Brookfield, CT (US); Yongda ZHANG, Sandy Hook, CT (US); Wenjun TANG, Southbury, CT (US)

(21) Appl. No.: 14/045,033

(22) Filed: Oct. 3, 2013

Related U.S. Application Data
(63) Continuation of application No. PCT/US2012/032027, filed on Apr. 3, 2012.
(60) Provisional application No. 61/481,894, filed on May 3, 2011.
(60) Provisional application No. 61/471,658, filed on Apr. 4, 2011.

(51) Int. Cl. C07D 491/06; C07D 417/02; C07D 409/02; C07D 401/02; C07D 215/14; C07D 405/02

(52) U.S. Cl. CPC C07D 491/06; C07D 215/14; C07D 405/02; C07D 409/02; C07D 401/02; C07D 417/02
USPC 546/89; 546/173; 546/99

(57) ABSTRACT

The present invention is directed to an improved process for the preparation of Compounds of Formula (I) or salts thereof which are useful in the treatment of HIV infection. In particular, the present invention is directed to an improved process for the preparation of (2S)-2-tert-butoxy-2-(4-(2,3-dihydropyrano[4,3,2-de]quinolin-7-yl)-2-methylquinolin-3-yl)acetic acid or salt thereof which is useful in the treatment of HIV infection.

### CROSS-REFERENCE TO RELATED APPLICATIONS

This application is a continuation of International PCT Patent Application No. PCT/US2012/032027, filed Apr. 3, 2012, now pending, which claims the benefit under 35 U.S.C. §119(e) of U.S. Provisional Patent Application No. 61/471,658, filed Apr. 4, 2011, and U.S. Provisional Patent Application No. 61/481,894, filed May 3, 2011, which applications are incorporated herein by reference in their entireties.

### BACKGROUND

1. Field

The present invention is directed to an improved process for the preparation of Compounds of Formula (I) or salts thereof which are useful in the treatment of HIV infection. In particular, the present invention is directed to an improved process for the preparation of (2S)-2-tert-butoxy-2-(4-(2,3-dihydropyrano[4,3,2-de]quinolin-7-y)-2-methylquinolin-3-yl)acetic acid (Compound 1001) or salts thereof which are useful in the treatment of HIV infection.

2. Description of the Related Art

Compounds of Formula (I) and salts thereof are known and potent inhibitors of HIV integrase:

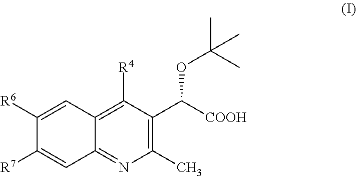

wherein:
R⁴ is selected from the group consisting of:

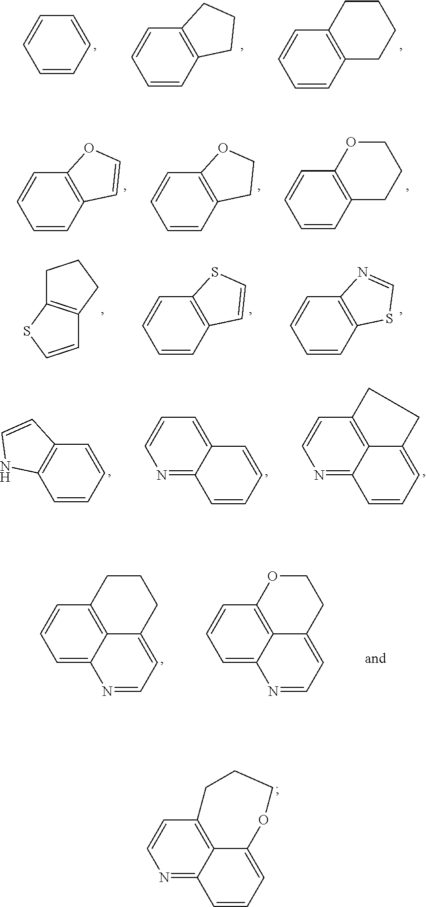

and
R⁶ and R⁷ are each independently selected from H, halo and (C₁₋₆)alkyl.

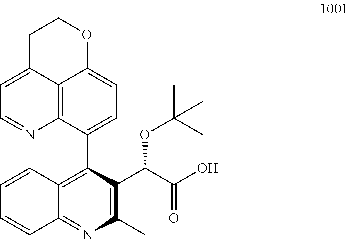

The compounds of Formula (I) and Compound 1001 fall within the scope of HIV inhibitors disclosed in WO 2007/131350. Compound 1001 is disclosed specifically as compound no. 1144 in WO 2009/062285. The compounds of Formula (I) and compound 1001 can be prepared according to the general procedures found in WO 2007/131350 and WO 2009/062285, which are hereby incorporated by reference.

The compounds of Formula (I) and Compound 1001 in particular have a complex structure and their synthesis is very challenging. Known synthetic methods face practical limitations and are not economical for large-scale production. There is a need for efficient manufacture of the compounds of Formula (I) and Compound 1001, in particular, with a minimum number of steps, good enantiomeric excess and sufficient overall yield. Known methods for production of the compounds of Formula (I) and Compound 1001, in particular, have limited yield of the desired atropisomer. There is lack of literature precedence as well as reliable conditions to achieve atropisomer selectivity. The present invention fulfills these needs and provides further related advantages.

### BRIEF SUMMARY

The present invention is directed to a synthetic process for preparing compounds of Formula (I), such as Compounds 1001-1055, using the synthetic steps described herein. The present invention is also directed to particular individual steps of this process and particular individual intermediates used in this process.

One aspect of the invention provides a process to prepare a compound of Formula (I) or a salt thereof:

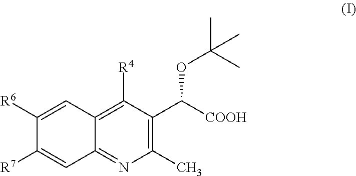

wherein:

R⁴ is selected from the group consisting of:

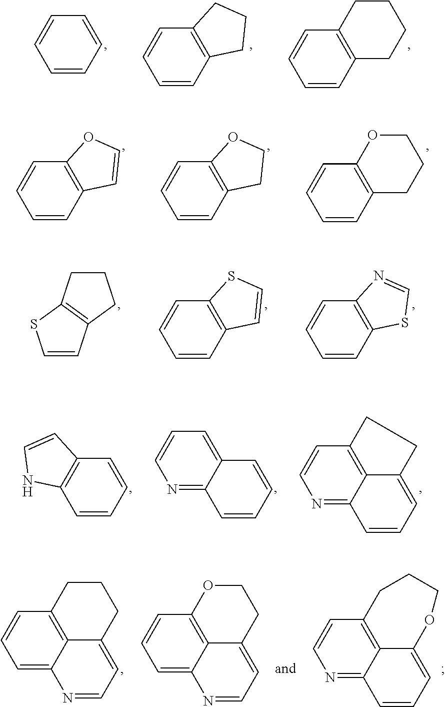

and
R⁶ and R⁷ are each independently selected from H, halo and (C₁₋₆)alkyl;
   in accordance with the following General Scheme I:

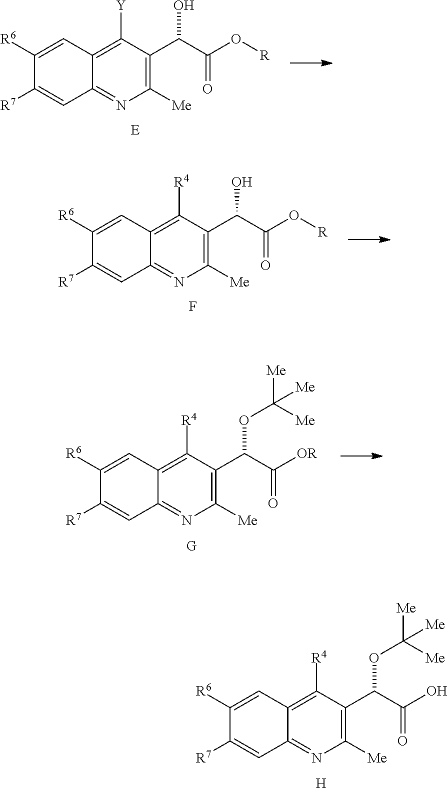

wherein:
Y is I, Br or Cl; and
R is (C₁₋₆)alkyl:
   wherein the process comprises:
coupling aryl halide E under diastereoselective Suzuki coupling conditions in the presence of a chiral biaryl monophosphorus ligand having Formula (AA):

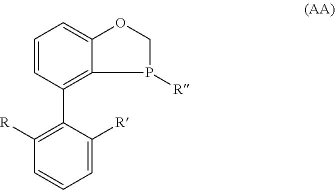

wherein R═R′═H; R″=tert-butyl; or R═OMe; R′═H; R″=tert-butyl; or R═N(Me)₂; R′═H; R″=tert-butyl;
   in combination with a palladium catalyst or precatalyst, and a base and a boronic acid or boronate ester in a solvent mixture;
converting chiral alcohol F to tert-butyl ether G under BrØnstead- or Lewis-acid catalysis with a source tert-butyl cation or its equivalent;
saponifying ester G to inhibitor H in a solvent mixture; and
optionally converting inhibitor H to a salt.

Another aspect of the invention provides a process to prepare a compound of Formula (I) or a salt thereof:

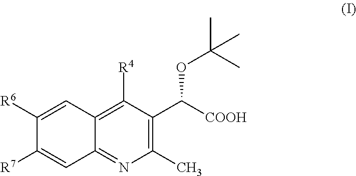

wherein:
R⁴ is selected from the group consisting of:

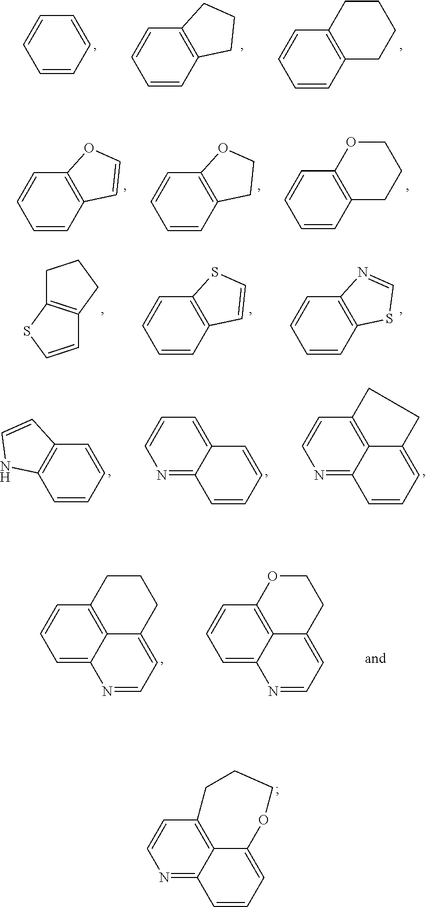

and
R⁶ and R⁷ are each independently selected from H, halo and (C₁₋₆)alkyl;
   in accordance with the following General Scheme I:

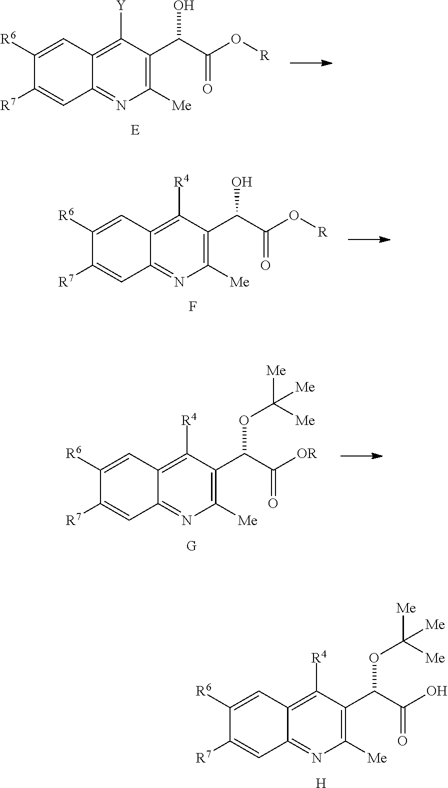

wherein:
Y is I, Br or Cl; and
R is (C₁₋₆)alkyl:
   wherein the process comprises:
subjecting aryl halide E to a diastereoselective Suzuki coupling reaction employing a chiral biaryl monophosphorus ligand having Formula (AA):

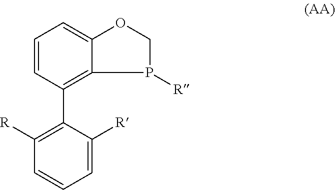

wherein R═R′═H; R″=tert-butyl; or R═OMe; R′═H; R″=tert-butyl; or R═N(Me)₂; R′═H; R″=tert-butyl;
   in combination with a palladium catalyst or precatalyst, a base and an appropriate boronic acid or boronate ester in an appropriate solvent mixture;
converting chiral alcohol F to tert-butyl ether G under BrØnstead- or Lewis-acid catalysis with a source tert-butyl cation or its equivalent;
converting ester G to an inhibitor H through a standard saponification reaction in a suitable solvent mixture; and
optionally converting the inhibitor H to a salt thereof using standard methods.

Another aspect of the invention provides a process to prepare a compound of Formula (I) or salt thereof:

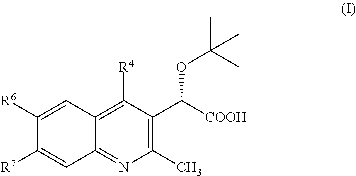

wherein:
R⁴ is selected from the group consisting of:

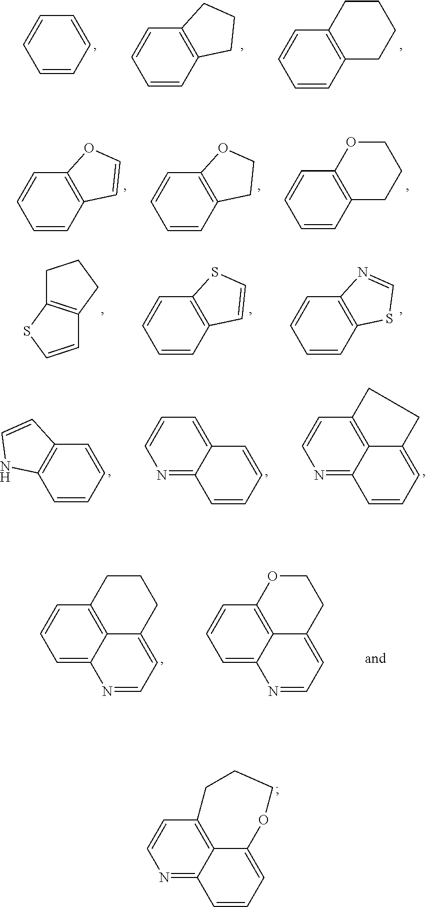

and
R⁶ and R⁷ are each independently selected from H, halo and (C₁₋₆)alkyl;
   in accordance with the following General Scheme II:

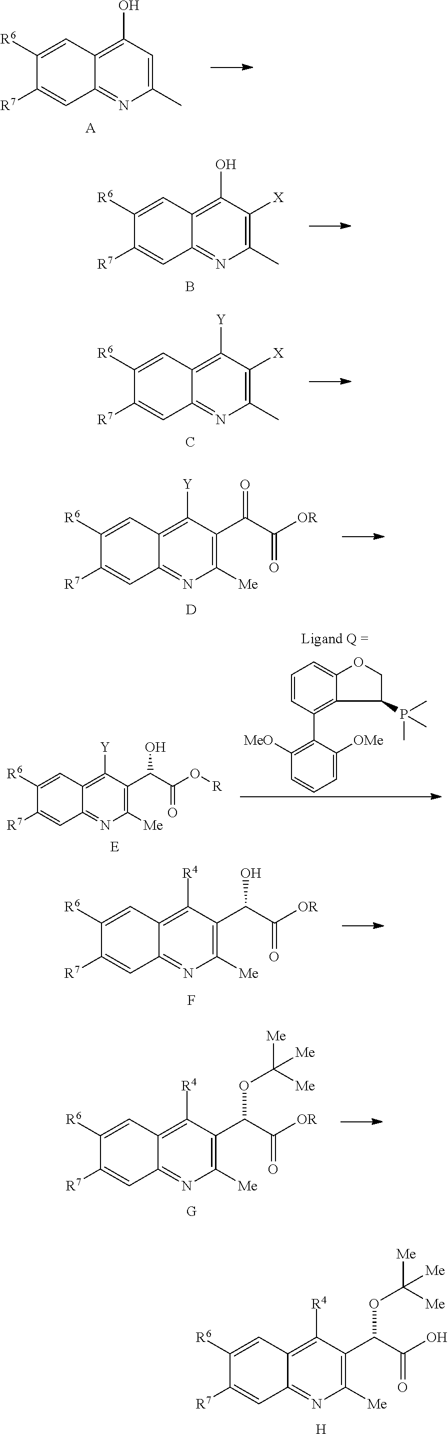

wherein:
X is I or Br.
Y is Cl when X is Br or I, or Y is Br when X is I, or Y is I; and
R is (C₁₋₆)alkyl;
   wherein the process comprises:
converting 4-hydroxyquinoline A to phenol B via a regioselective halogenation reaction at the 3-position of the quinoline core;
converting phenol B to aryl dihalide C through activation of the phenol with an activating reagent and subsequent treatment with a halide source in the presence of an organic base;
converting aryl dihalide C to ketone D by chemoselectively transforming the 3-halo group to an aryl metal reagent and then reacting the aryl metal reagent with an activated carboxylic acid;
stereoselectively reducing ketone D to chiral alcohol E by asymmetric ketone reduction methods;
diastereoselectively coupling of aryl halide E with R⁴ in the presence of phosphine ligand Q in combination with a palladium catalyst or precatalyst, a base and a boronic acid or boronate ester in a solvent mixture;
converting chiral alcohol F to tert-butyl ether G under BrØnstead- or Lewis-acid catalysis with a source tert-butyl cation or its equivalent;
saponifying ester G to inhibitor H in a solvent mixture; and
optionally converting inhibitor H to a salt thereof.

Another aspect of the invention provides a process to prepare a compound of Formula (I) or salt thereof:

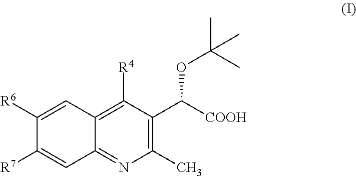

wherein:
R⁴ is selected from the group consisting of:

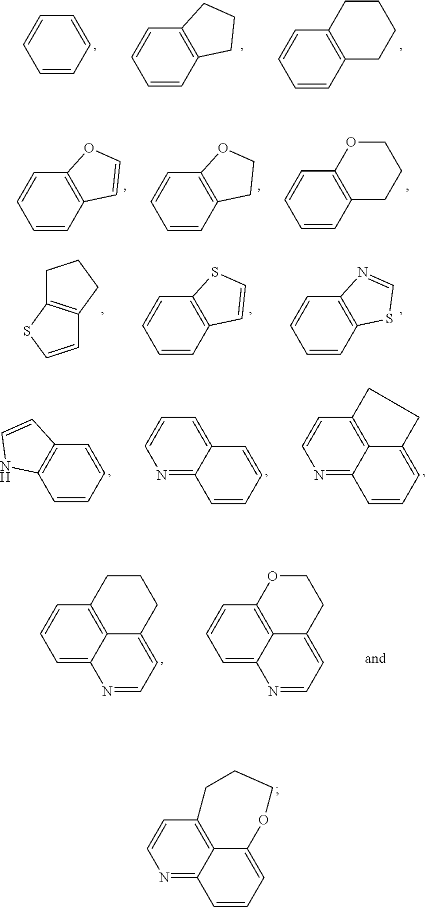

and

R⁶ and R⁷ are each independently selected from H, halo and (C₁₋₆)alkyl;

in accordance with the following General Scheme II:

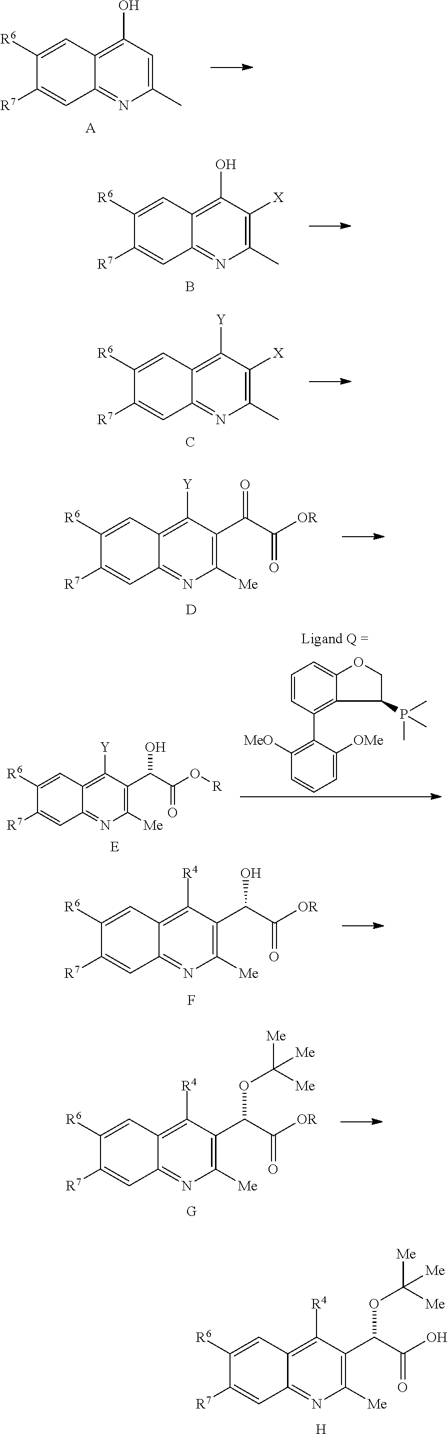

wherein:
X is I or Br;
Y is Cl when X is Br or I, or Y is Br when X is I, or Y is I; and
R is (C₁₋₆)alkyl;
   wherein the process comprises:
converting 4-hydroxyquinoline A to phenol B via a regioselective halogenation reaction at the 3-position of the quinoline core;
converting phenol B to aryl dihalide C through activation of the phenol with a suitable activating reagent and subsequent treatment with an appropriate halide source, in the presence of an organic base;
converting aryl dihalide C to ketone D by first chemoselective transformation of the 3-halo group to an aryl metal reagent, and then reaction of this intermediate with an activated carboxylic acid;
stereoselectively reducing ketone D to chiral alcohol E by standard asymmetric ketone reduction methods;
subjecting aryl halide E to a diastereoselective Suzuki coupling reaction employing chiral phosphine Q in combination with a palladium catalyst or precatalyst, a base and an appropriate boronic acid or boronate ester in an appropriate solvent mixture;
converting chiral alcohol F to tert-butyl ether G under BrØnstead- or Lewis-acid catalysis with a source tert-butyl cation or its equivalent;
converting ester G to an inhibitor H through a standard saponification reaction in a suitable solvent mixture; and
optionally converting the inhibitor H to a salt thereof using standard methods.

Another aspect of the invention provides a process to prepare Compounds 1001-1055 or a salt thereof in accordance with the above General Scheme I.

Another aspect of the invention provides a process to prepare Compounds 1001-1055 or a salt thereof in accordance with the above General Scheme II.

Another aspect of the invention provides a process for the preparation of Compound 1001 or a salt thereof,

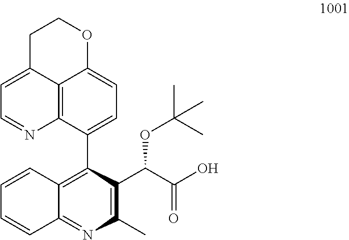

in accordance with the following General Scheme IA:

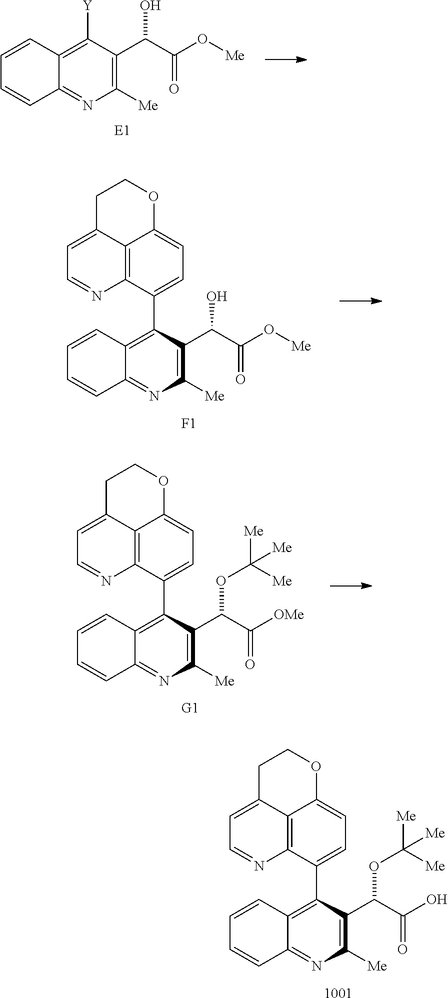

wherein Y is I, Br or Cl;
wherein the process comprises:
coupling aryl halide E1 under diastereoselective Suzuki coupling conditions in the presence of a chiral biaryl monophosphorus ligand having Formula (AA):

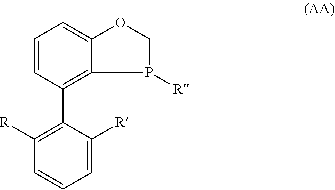

wherein R═R′═H; R″=tert-butyl; or R═OMe; R′═H; R″=tert-butyl; or R═N(Me)₂; R′═H; R″=tert-butyl;
   in combination with a palladium catalyst or precatalyst, and a base and a boronic acid or boronate ester in a solvent mixture:
converting chiral alcohol FI to tert-butyl ether G1 under BrØnstead- or Lewis-acid catalysis with a source tert-butyl cation or its equivalent;
saponifying ester G1 to Compound 1001 in a solvent mixture; and
optionally converting Compound 1001 to a salt.

Another aspect of the invention provides a process for the preparation of Compound 1001 or a salt thereof,

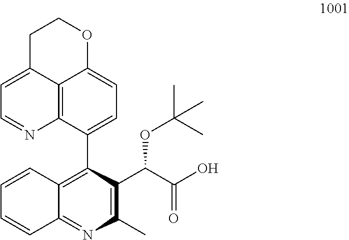

in accordance with the following General Scheme IA:

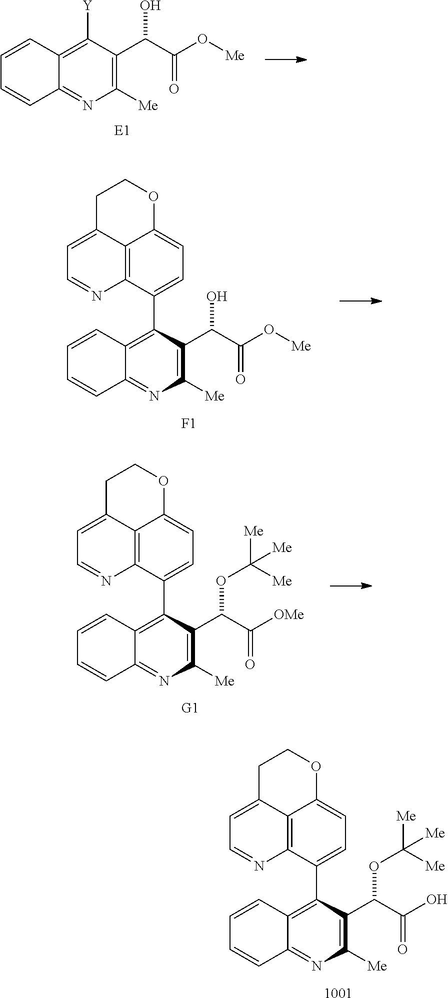

wherein Y is I, Br or Cl;
wherein the process comprises:
subjecting aryl halide E1 to a diastereoselective Suzuki coupling reaction employing a chiral biaryl monophosphorus ligand having Formula (AA):

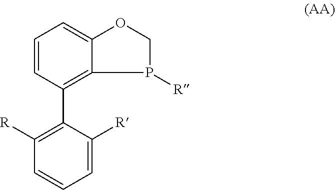

wherein R═R′═H; R″=tert-butyl; or R═OMe; R′═H; R″=tert-butyl; or R═N(Me); R′═H; R″=tert-butyl;
   in combination with a palladium catalyst or precatalyst, a base and an appropriate boronic acid or boronate ester in an appropriate solvent mixture;
converting chiral alcohol F1 to tert-butyl ether G1 under BrØnstead- or Lewis-acid catalysis with a source tert-butyl cation or its equivalent;
converting ester G1 to Compound 1001 through a standard saponification reaction in a suitable solvent mixture; and
optionally converting Compound 1001 to a salt thereof using standard methods.

Another aspect of the present invention provides a process for the preparation of Compound 1001 or salt thereof:

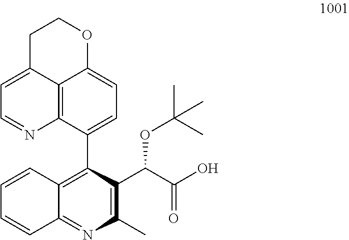

in accordance with the following General Scheme IIA:

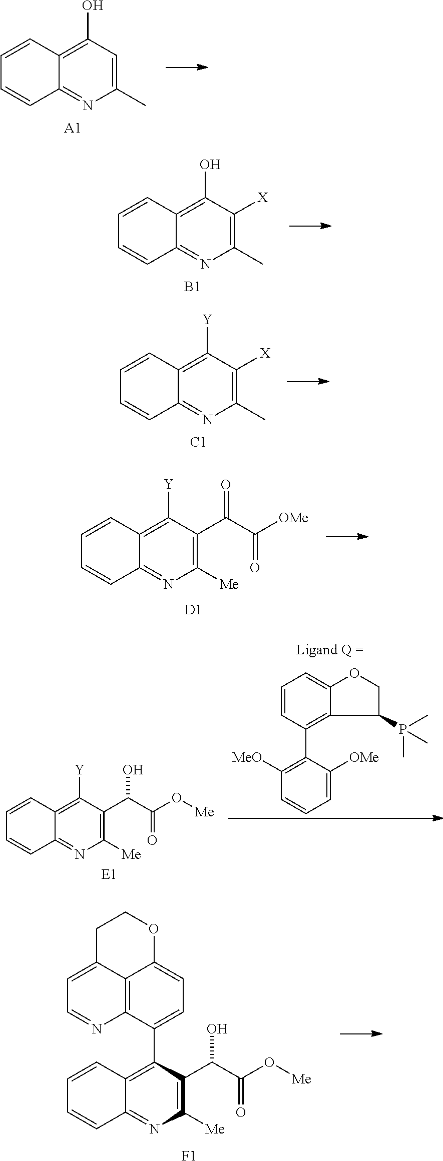

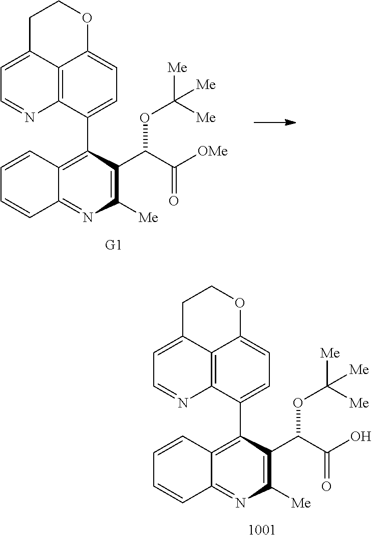

wherein:
X is I or Br, and
Y is Cl when X is Br or I, or Y is Br when X is I, or Y is I;
   wherein the process comprises:
converting 4-hydroxyquinoline A1 to phenol B1 via a regioselective halogenation reaction at the 3-position of the quinoline core;
converting phenol B1 to aryl dihalide C1 through activation of the phenol with an activating reagent and subsequent treatment with a halide source in the presence of an organic base;
converting aryl dihalide C1 to ketone D1 by chemoselectively transforming the 3-halo group to an aryl metal reagent and then reacting the aryl metal reagent with an activated carboxylic acid;
stereoselectively reducing ketone D1 to chiral alcohol E1 by asymmetric ketone reduction methods;
diastereoselectively coupling aryl halide E1 under Suzuki coupling reaction conditions in the presence of a chiral phosphine ligand Q in combination with a palladium catalyst or precatalyst, a base and a boronic acid or boronate ester in a solvent mixture;
converting chiral alcohol F1 to tert-butyl ether G1 under BrØnstead- or Lewis-acid catalysis with a source tert-butyl cation or its equivalent;
saponifying ester G1 to Compound 1001 in a solvent mixture; and
optionally converting Compound 1001 to a salt thereof.

Another aspect of the present invention provides a process for the preparation of Compound 1001 or salt thereof:

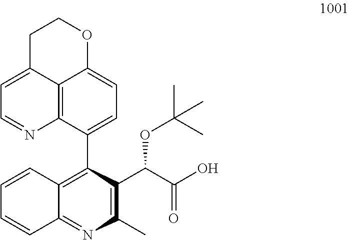

in accordance with the following General Scheme IIA:

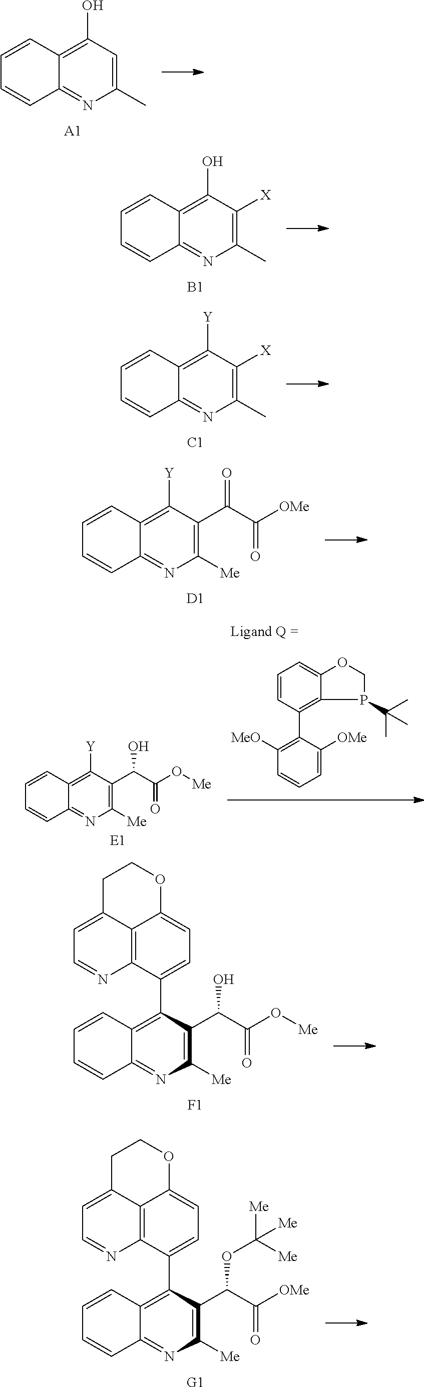

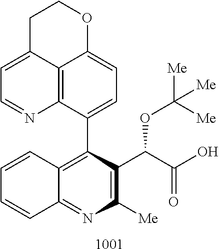

wherein:
X is I or Br; and
Y is Cl when X is Br or I, or Y is Br when X is I, or Y is I;
   wherein the process comprises:
converting 4-hydroxyquinoline A1 to phenol B1 via a regioselective halogenation reaction at the 3-position of the quinoline core;
converting phenol B1 to aryl dihalide C1 through activation of the phenol with a suitable activating reagent and subsequent treatment with an appropriate halide source, in the presence of an organic base;
converting aryl dihalide C1 to ketone D1 by first chemoselective transformation of the 3-halo group to an aryl metal reagent, and then reaction of this intermediate with an activated carboxylic acid;
stereoselectively reducing ketone D1 to chiral alcohol E1 by standard asymmetric ketone reduction methods;
subjecting aryl halide E1 to a diastereoselective Suzuki coupling reaction employing chiral phosphine Q in combination with a palladium catalyst or precatalyst, a base and an appropriate boronic acid or boronate ester in an appropriate solvent mixture;
converting chiral alcohol F1 to tert-butyl ether G1 under BrØnstead- or Lewis-acid catalysis with a source tert-butyl cation or its equivalent;
converting ester G1 to Compound 1001 through a standard saponification reaction in a suitable solvent mixture; and
optionally converting Compound 1001 to a salt thereof using standard methods.

Another aspect of the present invention provides a process for the preparation of a quinoline-8-boronic acid derivative or a salt thereof in accordance with the following General Scheme III:

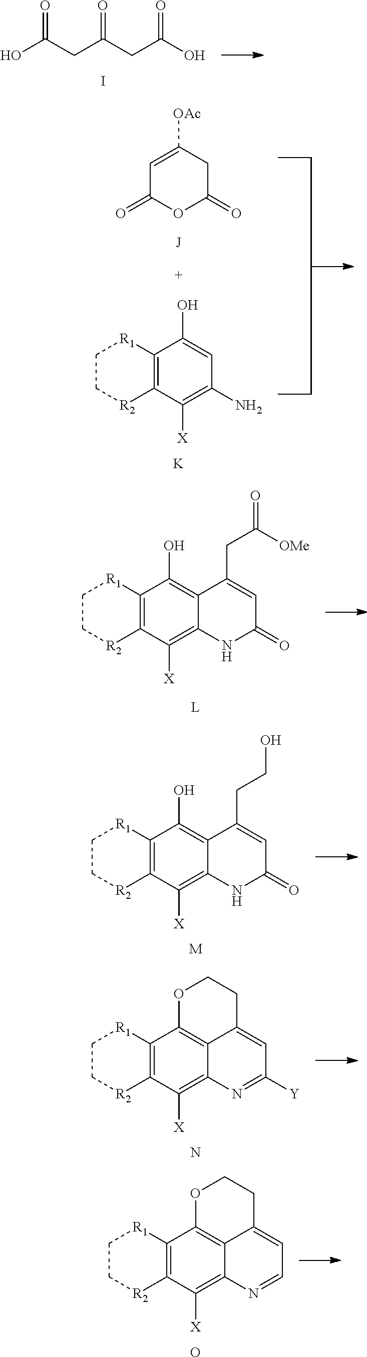

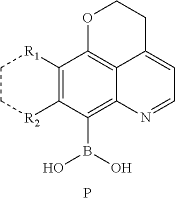

wherein:
X is Br or I;
Y is Br or Cl; and
R₁ and R₂ may either be absent or linked to form a cycle;
   wherein the process comprises:
converting diacid I to cyclic anhydride J;
condensing anhydride J with meta-aminophenol K to give quinolone L;
reducing the ester of compound L to give alcohol M;
cyclizing alcohol M to give tricyclic quinoline N by activating the alcohol as its corresponding alkyl chloride or alkyl bromide;
reductively removing halide Y under acidic conditions in the presence of a reductant to give compound O;
converting halide X in compound O to the corresponding boronic acid P, sequentially via the corresponding intermediate aryl lithium reagent and boronate ester; and
optionally converting compound P to a salt thereof.

Another aspect of the present invention provides a process for the preparation of a quinoline-8-boronic acid derivative or a salt thereof in accordance with the following General Scheme III:

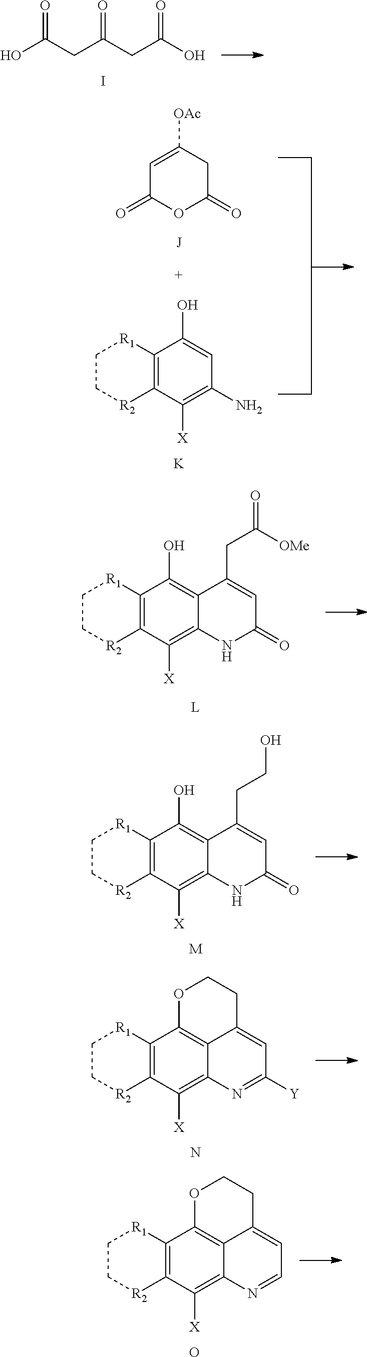

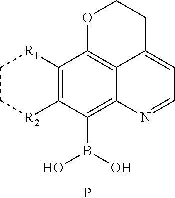

wherein:
X is Br or I;
Y is Br or Cl; and
R₁ and R₂ may either be absent or linked to form a cycle;
   wherein the process comprises:
converting diacid I to cyclic anhydride J under standard conditions;
condensing anhydride J with meta-aminophenol K to give quinolone L;
reducing the ester of compound L under standard conditions to give alcohol M, which then undergoes a cyclization reaction to give tricyclic quinoline N via activation of the alcohol as its corresponding alkyl chloride or alkyl bromide;
reductive removal of halide Y is achieved under acidic conditions with a reductant to give compound O;
converting halide X in compound O to the corresponding boronic acid P, sequentially via the corresponding intermediate aryl lithium reagent and boronate ester; and
optionally converting compound P to a salt thereof using standard methods.

Another aspect of the present invention provides a process for the preparation of Compound 1001 or salt thereof in accordance with General Scheme III and General Scheme IA.

Another aspect of the present invention provides a process for the preparation of Compound 1001 or salt thereof in accordance with General Scheme III and General Scheme IIA.

Another aspect of the present invention provides novel intermediates useful in the production of Compound of Formula (I) or Compound 1001. In a representative embodiment, the invention provides one or more intermediates selected from:

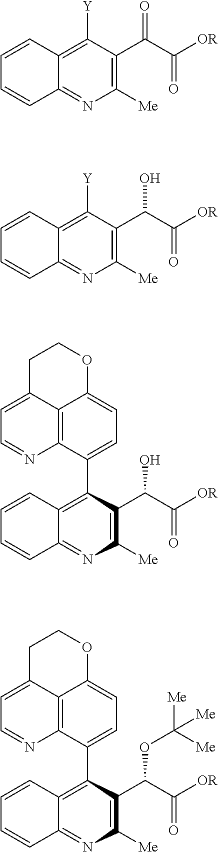

wherein:
Y is Cl, Br or I; and
R is (C₁₋₆)alkyl.

Further objects of this invention arise for the one skilled in the art from the following description and the examples.

### DETAILED DESCRIPTION

### Definitions

Terms not specifically defined herein should be given the meanings that would be given to them by one of skill in the art in light of the disclosure and the context. As used throughout the present application, however, unless specified to the contrary, the following terms have the meaning indicated:

Compound 1001, (2S)-2-tert-butoxy-2-(4-(2,3-dihydropyrano[4,3,2-de]quinolin-7-yl)-2-methylquinolin-3-yl)acetic acid:

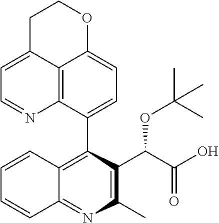

may alternatively be depicted as:

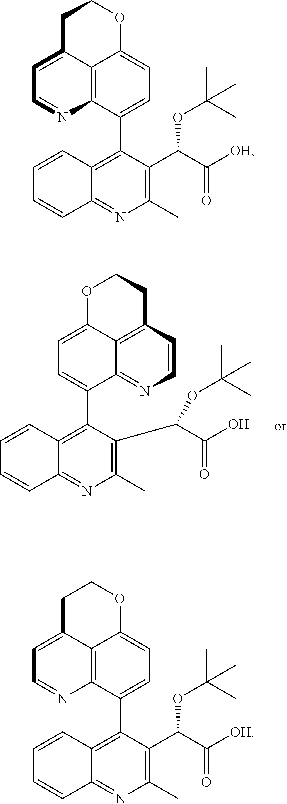

In addition, as one of skill in the art would appreciate, Compound (I) may alternatively be depicted in a zwitterionic form.

The term “precatalyst” means active bench stable complexes of a metal (such as, palladium) and a ligand (such as a chiral biaryl monophorphorus ligand or chiral phosphine ligand) which are easily activated under typical reaction conditions to give the active form of the catalyst. Various precatalysts are commercially available.

The term tert-butyl cation “equivalent” Includes tertiary carbocations such as, for example, tert-butyl-2,2,2-trichloroacetimidate, 2-methylpropene, tert-butanol, methyl tert-butylether, tert-butylacetate and tert-butyl halide (halide could be chloride, bromide and iodide).

The term “halo” or “halide” generally denotes fluorine, chlorine, bromine and iodine.

The term “(C₁₋₆)alkyl” wherein n is an integer from 2 to n, either alone or in combination with another radical denotes an acyclic, saturated, branched or linear hydrocarbon radical with I to n C atoms. For example the term (C₁₋₃)alkyl embraces the radicals H₃C—, H₃C—CH₂—, H₃C—CH₂—CH₂— and H₃C—CH(CH₃)—.

The term “carbocyclyl” or “carbocycle” as used herein, either alone or in combination with another radical, means a mono-, bi- or tricyclic ring structure consisting of 3 to 14 carbon atoms. The term “carbocycle” refers to fully saturated and aromatic ring systems and partially saturated ring systems. The term “carbocycle” encompasses fused, bridged and spirocyclic systems.

The term “aryl” as used herein, either alone or in combination with another radical, denotes a carbocyclic aromatic monocyclic group containing 6 carbon atoms which may be further fused to at least one other 5- or 6-membered carbocyclic group which may be aromatic, saturated or unsaturated. Aryl includes, but is not limited to, phenyl, indanyl, indenyl, naphthyl, anthracenyl, phenanthrenyl, tetrahydronaphthyl and dihydronaphthyl.

The terms “boronic acid” or “boronic acid derivative” refer to a compound containing the —B(OH)₂ radical. The terms “boronic ester” or “boronic ester derivative” refer to a compound containing the —B(OR)(OR′) radical, wherein each of R and R′, are each independently alkyl or wherein R and R′ join together to form a heterocyclic ring. Selected examples of the boronic acids or boronate esters that may be used are, for example:

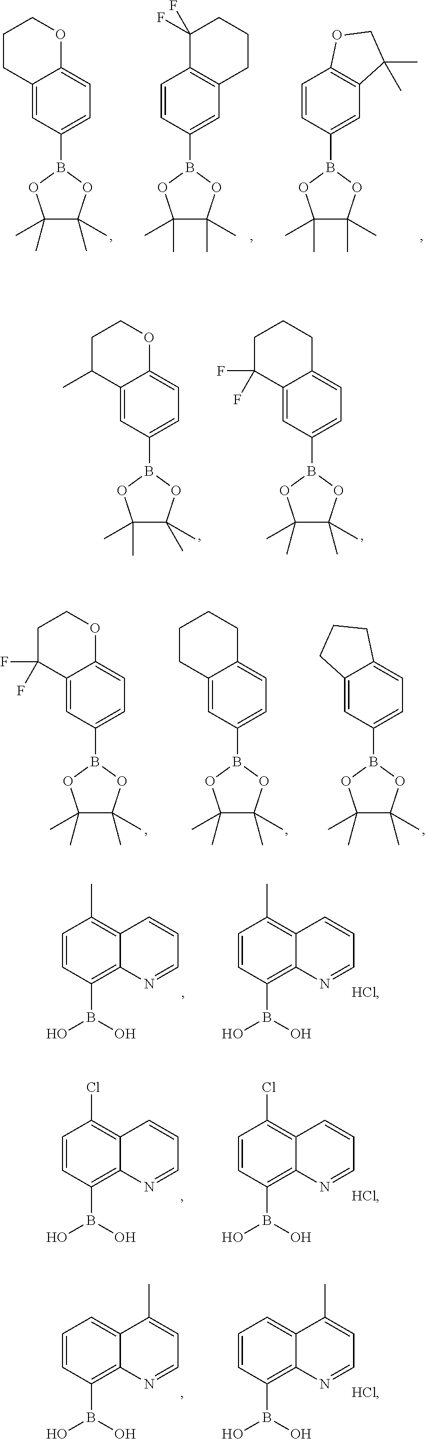

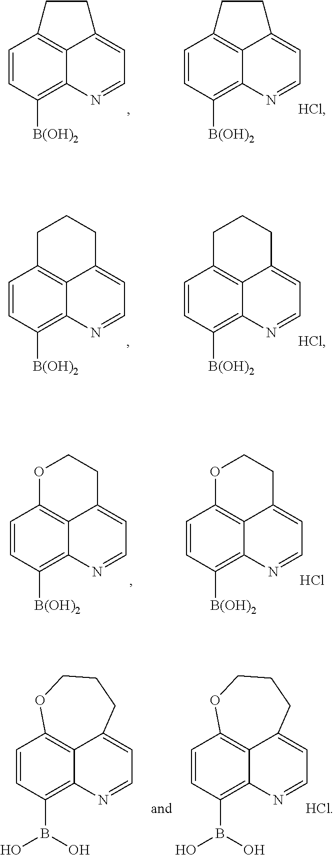

“Heterocyclyl” or “heterocyclic ring” refers to a stable 3- to 18-membered non-aromatic ring radical which consists of two to twelve carbon atoms and from one to six heteroatoms selected from the group consisting of nitrogen, oxygen, sulfur and boron. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quatemized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

The following designation [symbol] is used in sub-formulas to indicate the bond which is connected to the rest of the molecule as defined.

The term “salt thereof” as used herein is intended to mean any acid and/or base addition salt of a compound according to the invention, including but not limited to a pharmaceutically acceptable salt thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. For example, such salts include acetates, ascorbates, benzenesulfonates, benzoates, besylates, bicarbonates, bitartrates, bromides/hydrobromides, Ca-edetates/edetates, camsylates, carbonates, chlorides/hydrochlorides, citrates, edisylates, ethane disulfonates, estolates esylates, fumarates, gluceptates, gluconates, glutamates, glycolates, glycollylarsnilates, hexyiresorcinates, hydrabamines, hydroxymaleates, hydroxynaphthoates, iodides, isothionates, lactates, lactobionates, malates, maleates, mandelates, methanesulfonates, mesylates, methylbromides, methylnitrates, methylsulfates, mucates, napsylates, nitrates, oxalates, pamoates, pantothenates, phenylacetates, phosphates/diphosphates, polygalacturonates, propionates, salicylates, stearates subacetates, succinates, sulfamides, sulfates, tannates, tartrates, teoclates, toluenesulfonates, triethiodides, ammonium, benzathines, chloroprocaines, cholines, diethanolamines, ethylenediamines, meglumines and procaines. Further pharmaceutically acceptable salts can be formed with cations from metals like aluminium, calcium, lithium, magnesium, potassium, sodium, zinc and the like. (also see Pharmaceutical salts, Birge, S. M. et al., J. Pharm. Sci., (1977), 66, 1-19).

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent like ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts) also comprise a part of the invention.

The term “treating” with respect to the treatment of a disease-state in a patient include (i) inhibiting or ameliorating the disease-state in a patient, e.g., arresting or slowing its development; or (ii) relieving the disease-state in a patient, i.e., causing regression or cure of the disease-state. In the case of HIV, treatment includes reducing the level of HIV viral load in a patient.

The term “antiviral agent” as used herein is intended to mean an agent that is effective to inhibit the formation and/or replication of a virus in a human being, including but not limited to agents that interfere with either host or viral mechanisms necessary for the formation and/or replication of a virus in a human being. The term “antiviral agent” includes, for example, an HIV integrase catalytic site inhibitor selected from the group consisting: raltegravir (ISENTRESS®; Merck); elvitegravir (Gilead); soltegravir (GSK; ViiV); and GSK 1265744 (GSK; ViiV); an HIV nucleoside reverse transcriptase inhibitor selected from the group consisting of: abacavir (ZIAGEN®; GSK); didanosine (VIDEX®; BMS); tenofovir (VIREAD®; Gilead); emtricitabine (EMTRIVA®; Gilead); lamivudine (EPIVIR®; GSK/Shire); stavudine (ZERIT®; BMS); zidovudine (RETROVIR®; GSK); elvucitabine (Achillion); and festinavir (Oncolys); an HIV non-nucleoside reverse transcriptase inhibitor selected from the group consisting oft nevirapine (VIRAMUNE®; BI); efavirenz (SUSTIVA®; BMS); etravirine (INTELENCE®; J&J); rilpivirine (TMC278, R278474; J&J): fosdevirine (GSK/ViiV); and lersivirine (Pfizer/ViiV); an HIV protease inhibitor selected from the group consisting of: atazanavir (REYATAZ®; BMS); darunavir (PREZISTA®; J&J); indinavir (CRIXIVAN®; Merck); lopinavir (KELETRA®; Abbott); nelfinavir (VIRACEPT®; Pfizer); saquinavir (INVIRASE; Hoffmann-LaRoche); tipranavir (APTIVUS®; BI); ritonavir (NORVIR®, Abbott); and fosamprenavir (LEXIVA®; GSK/Vertex); an HIV entry inhibitor selected from: maraviroc (SELZENTRY®; Pfizer); and enfuvirtide (FUZEON®; Trimeris); and an HIV maturation inhibitor selected from: bevirirnat (Myriad Genetics).

The term “therapeutically effective amount” means an amount of a compound according to the invention, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

### Representative Embodiments

In the synthetic schemes below, unless specified otherwise, all the substituent groups in the chemical formulas shall have the meanings as in Formula (I). The reactants used in the examples below may be obtained either as described herein, or if not described herein, are themselves either commercially available or may be prepared from commercially available materials by methods known in the art. Certain starting materials, for example, may be obtained by methods described in the International Patent Applications WO 2007/131350 and WO 2009/062285. Optimum reaction conditions and reaction times may vary depending upon the particular reactants used. Unless otherwise specified, solvents, temperatures, pressures, and other reaction conditions may be readily selected by one of ordinary skill in the art. Typically, reaction progress may be monitored by High Pressure Liquid Chromatography (HPLC), if desired, and intermediates and products may be purified by chromatography on silica gel and/or by recrystallization.

In one embodiment, the present invention is directed to the multi-step synthetic method for preparing compounds of Formula (I) and, in particular, Compounds 1001-1055, as set forth in Schemes I and II. In another embodiment, the present invention is directed to the multi-step synthetic method for preparing Compound 1001 as set forth in Schemes IA, IIA, and III. In other embodiments, the invention is directed to each of the individual steps of Schemes I, II, IA, IIA and III and any combination of two or more successive steps of Schemes I, II, IA, IIA and III.

### I. General Scheme I—General Multi-Step Synthetic Method to Prepare Compounds of Formula (I), or Salts Thereof, in Particular Compounds 1001-1055 or Salts Thereof

In one embodiment, the present invention is directed to a general multi-step synthetic method for preparing Compounds of Formula (I) or a salt thereof, in particular. Compounds 1001-1055 or a salt thereof:

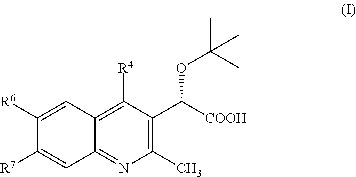

wherein:
R⁴ is selected from the group consisting of:

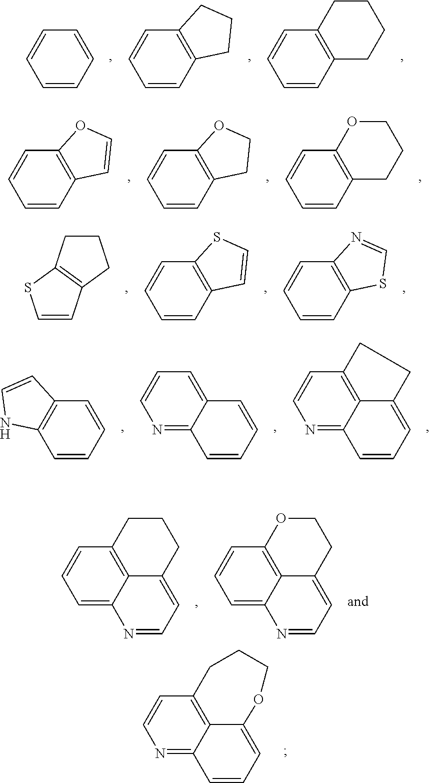

and
R⁶ and R⁷ are each independently selected from H, halo and (C₁₋₆)alkyl;
   according to the following General Scheme I:

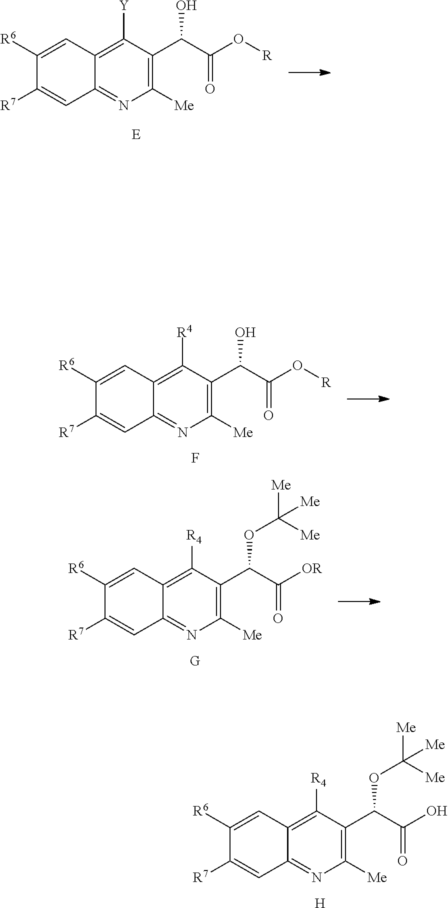

wherein:
Y is I, Br or Cl; and
R is (C₁₋₆)alkyl;
   wherein the process comprises:
coupling aryl halide E under diastereoselective Suzuki coupling conditions in the presence of a chiral biaryl monophosphorus ligand having Formula (AA):

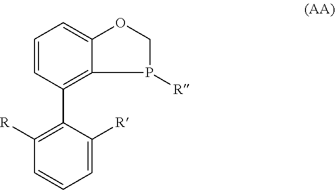

wherein R═R′═H; R″=tert-butyl; or R═OMe; R′═H; R″=tert-butyl; or R═N(Me)₂; R′═H; R″=tert-butyl;
   in combination with a palladium catalyst or precatalyst, and a base and a boronic acid or boronate ester in a solvent mixture;
converting chiral alcohol F to tert-butyl ether G under BrØnstead- or Lewis-acid catalysis with a source tert-butyl cation or its equivalent;
saponifying ester G to inhibitor H in a solvent mixture; and
optionally converting inhibitor H to a salt.

In another embodiment, the present invention is directed to a general multi-step synthetic method for preparing Compounds of Formula (I) or a salt thereof, in particular, Compounds 1001-1055 or a salt thereof:

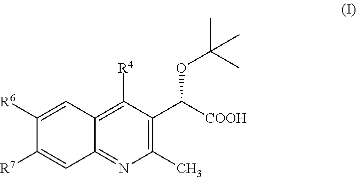

wherein:
R⁴ is selected from the group consisting of:

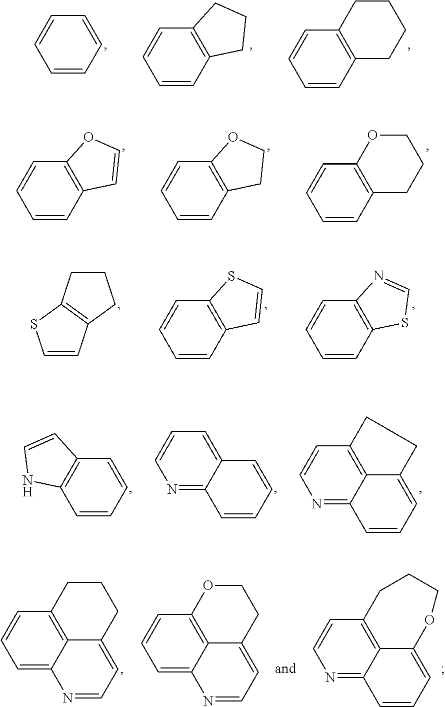

and
R⁶ and R⁷ are each independently selected from H, halo and (C₁₋₆)alkyl;
   according to the following General Scheme 1:

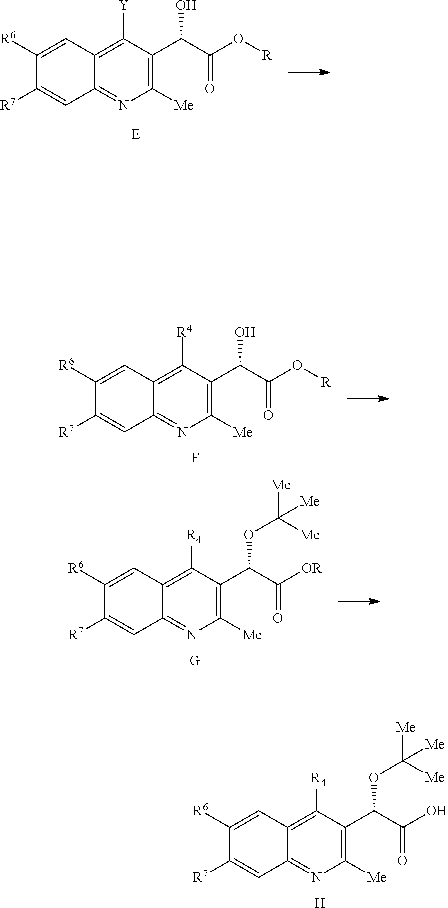

wherein:
Y is I, Br or Cl; and
R is (C₁₋₆)alkyl;
   wherein the process comprises:
subjecting aryl halide E to a diastereoselective Suzuki coupling reaction employing a chiral biaryl monophosphorus ligand having Formula (AA):

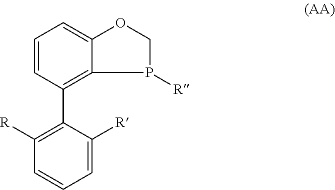

wherein R═R′═H; R″=tert-butyl; or R═OMe; R′═H; R″=tert-butyl; or R═N(Me)₂; R′═H; R″=tert-butyl;
   in combination with a palladium catalyst or precatalyst, a base and an appropriate boronic acid or boronate ester in an appropriate solvent mixture;
converting chiral alcohol F to tert-butyl ether G under BrØnstead- or Lewis-acid catalysis with a source tert-butyl cation or its equivalent;
converting ester G to an inhibitor H through a standard saponification reaction in a suitable solvent mixture; and
optionally converting the inhibitor H to a salt thereof using standard methods.

A person of skill in the art will recognize that the particular boronic acid or boronate ester will depend upon the desired R⁴ in the final inhibitor H. Selected examples of the boronic acid or boronate ester that may be used are, for example:

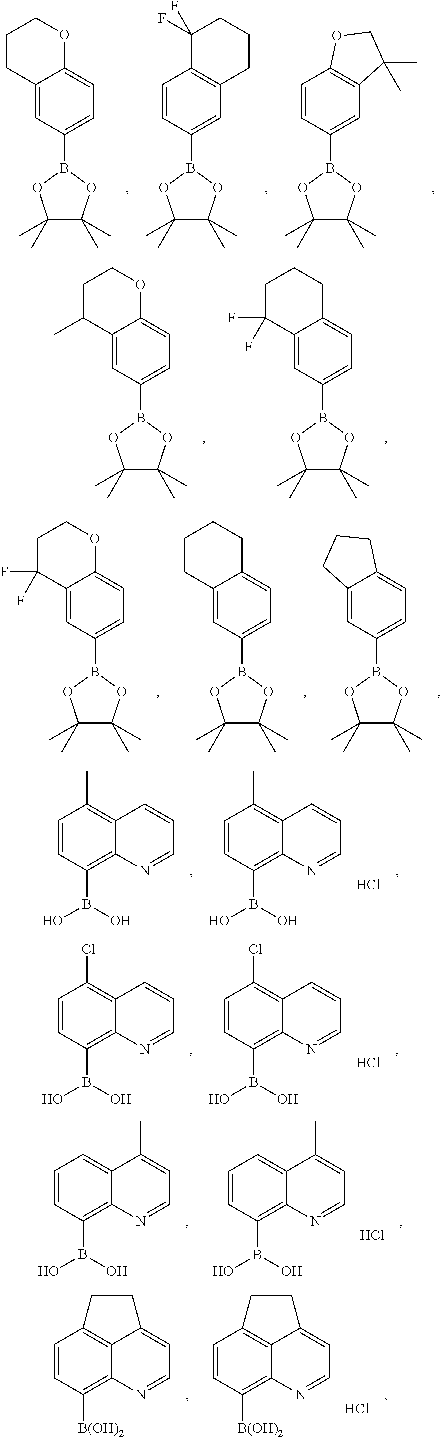

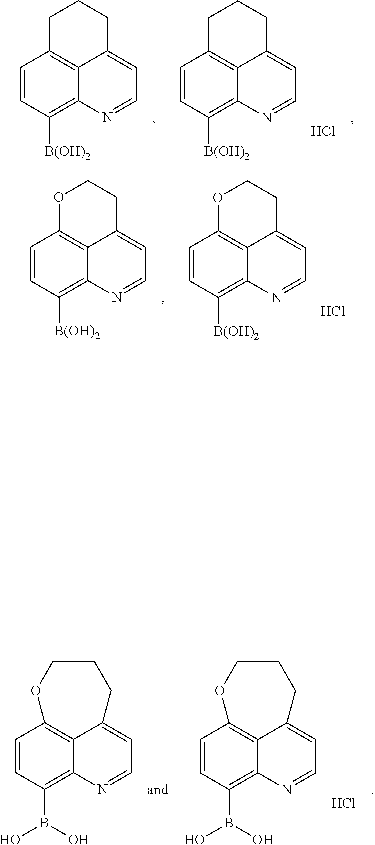

### II. General Scheme II—General Multi-Step Synthetic Method to Prepare Compounds of Formula (I), or Salts Thereof, in Particular Compounds 1001-1055 or Salts Thereof

In one embodiment, the present invention is directed to a general multi-step synthetic method for preparing Compounds of Formula (I) or a salt thereof, in particular, Compounds 1001-1055 or a salt thereof:

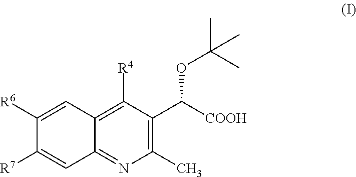

wherein:
R⁴ is selected from the group consisting of:

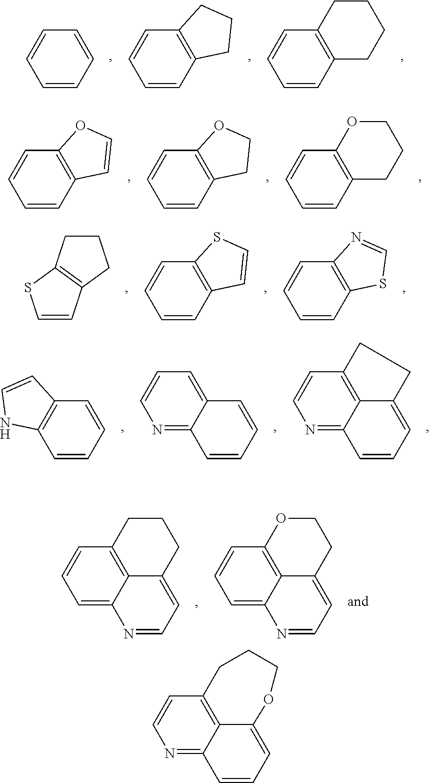

R⁶ and R⁷ are each independently selected from H, halo and (C₁₋₆)alkyl;
   according to the following General Scheme II:

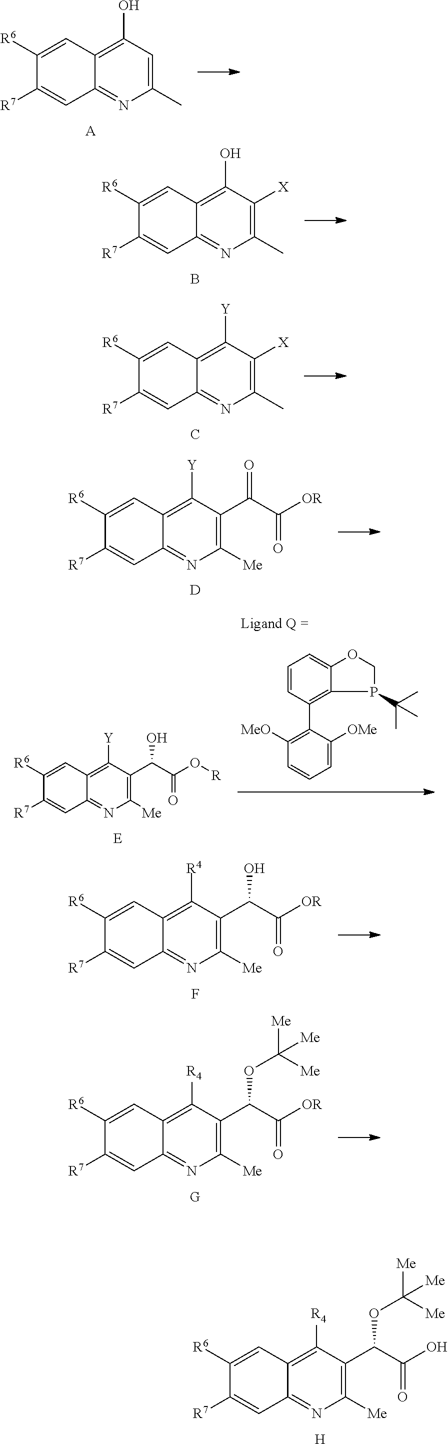

wherein:
X is I or Br;
Y is Cl when X is Br or I, or Y is Br when X is I, or Y is I; and
R is (C₁₋₆)alkyl;
   wherein the process comprises:
converting 4-hydroxyquinoline A to phenol B via a regioselective halogenation reaction at the 3-position of the quinoline core;
converting phenol B to aryl dihalide C through activation of the phenol with an activating reagent and subsequent treatment with a halide source in the presence of an organic base;
converting aryl dihalide C to ketone D by chemoselectively transforming the 3-halo group to an aryl metal reagent and then reacting the aryl metal reagent with an activated carboxylic acid;
stereoselectively reducing ketone D to chiral alcohol E by asymmetric ketone reduction methods;
diastereoselectively coupling aryl halide E with R⁴ under Suzuki coupling reaction conditions in the presence of a chiral phosphine ligand Q in combination with a palladium catalyst or precatalyst, a base and a boronic acid or boronate ester in a solvent mixture;
converting chiral alcohol F to tert-butyl ether G under BrØnstead- or Lewis-acid catalysis with a source tert-butyl cation or its equivalent;
saponifying ester G to inhibitor H in a solvent mixture: and
optionally converting inhibitor H to a salt thereof.

In one embodiment, the present invention is directed to a general multi-step synthetic method for preparing Compounds of Formula (I) or a salt thereof, in particular, Compounds 1001-1055 or a salt thereof:

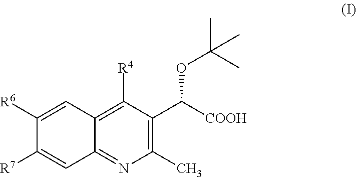

wherein:
R⁴ is selected from the group consisting of:

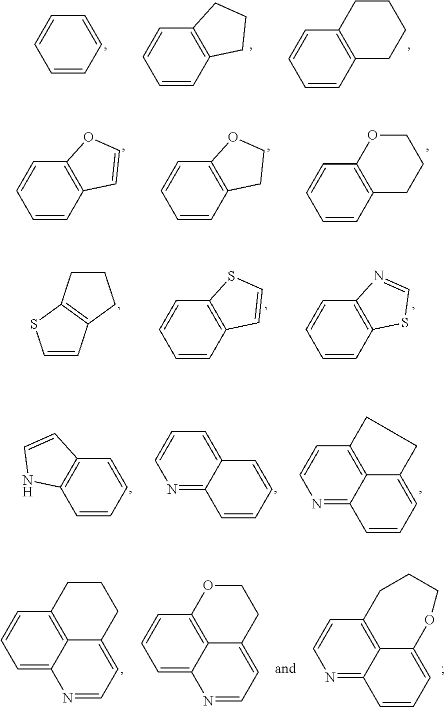

and
R⁶ and R⁷ are each independently selected from H, halo and (C₁₋₆)alkyl;
   according to the following General Scheme II:

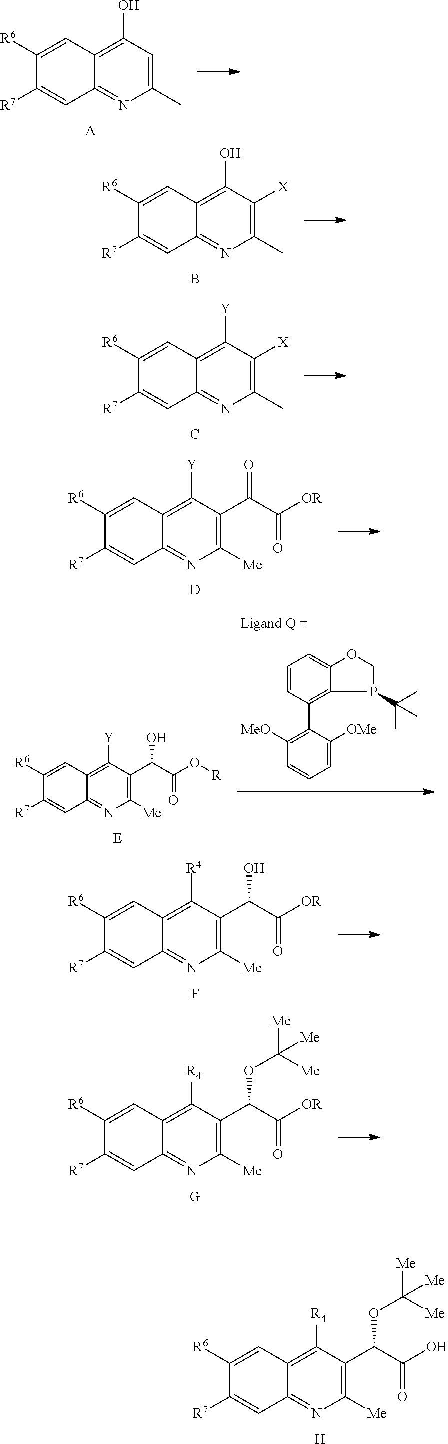

wherein:
X is I or Br,
Y is Cl when X is Br or I, or Y is Br when X is I, or Y is I; and
R is (C₁₋₆)alkyl;
   wherein the process comprises:
converting 4-hydroxyquinoline A to phenol B via a regioselective halogenation reaction at the 3-position of the quinoline core;
converting phenol B to aryl dihalide C through activation of the phenol with a suitable activating reagent and subsequent treatment with an appropriate halide source in the presence of an organic base;
converting aryl dihalide C to ketone D by first chemoselective transformation of the 3-halo group to an aryl metal reagent, and then reaction of this intermediate with an activated carboxylic acid;
stereoselectively reducing ketone D to chiral alcohol E by standard asymmetric ketone reduction methods;
subjecting aryl halide E to a diastereoselective Suzuki coupling reaction employing chiral phosphine Q in combination with a palladium catalyst or precatalyst, a base and an appropriate boronic acid or boronate ester in an appropriate solvent mixture;
converting chiral alcohol F to tert-butyl ether G under BrØnstead- or Lewis-acid catalysis with a source tert-butyl cation or its equivalent;
converting ester G to an inhibitor H through a standard saponification reaction in a suitable solvent mixture; and
optionally converting the inhibitor H to a salt thereof using standard methods.

A person of skill in the art will recognize that the particular boronic acid or boronate ester will depend upon the desired R⁴ in the final inhibitor H. Selected examples of the boronic acid or boronate ester that may be used are, for example:

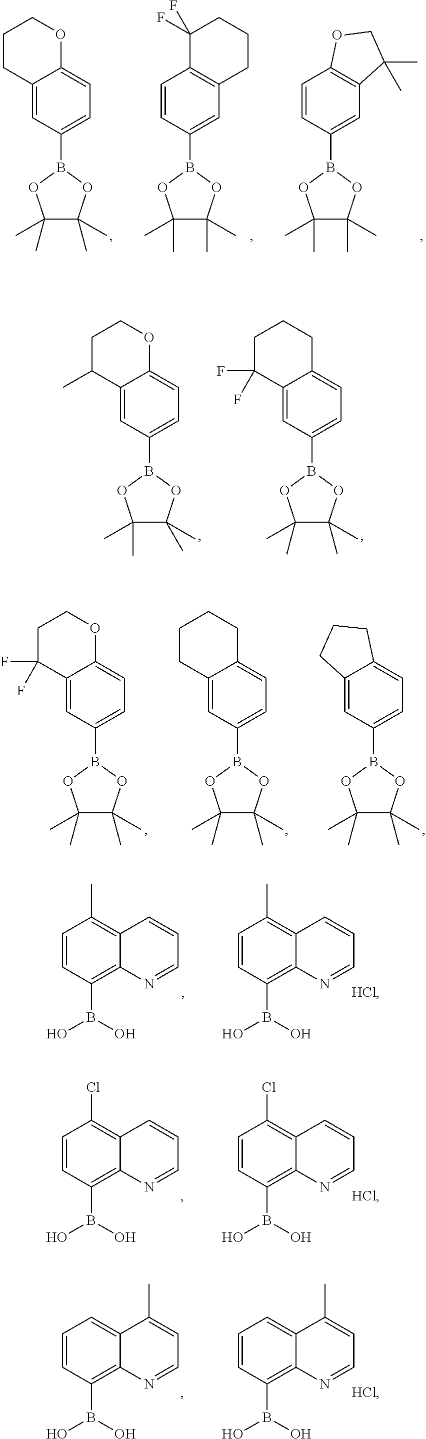

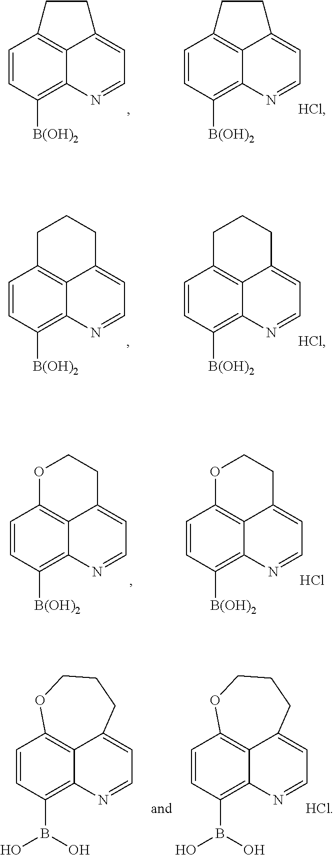

### III. General Schemes I and II—Individual Steps of the Synthetic Methods to Prepare Compounds of Formula (I) or Salts Thereof, in Particular Compounds 1001-1055 or Salts Thereof

Additional embodiments of the invention are directed to the individual steps of the multistep general synthetic methods described above in Sections I and II, namely General Schemes I and II, and the individual intermediates used in these steps. These individual steps and intermediates of the present invention are described in detail below. All substituent groups in the steps described below are as defined in the multi-step method above.

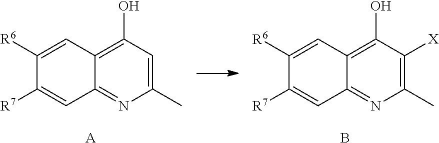

Readily or commercially available 4-hydroxyquinolines of general structure A are converted to phenol B via a regioselective halogenation reaction at the 3-position of the quinoline core. This may be accomplished with electrophilic halogenation reagents known to those of skill in the art, such as, for example, but not limited to NIS, NBS, I₂, NaI/I₂, Br₂, Br—I, Cl—I or Br₃ pyr. Preferably, 4-hydroxyquinolines of general structure A are converted to phenol B via a regioselective iodination reaction at the 3-position of the quinoline core. More preferably, 4-hydroxyquinolines of general structure A are converted to phenol B via a regioselective iodination reaction at the 3-position of the quinoline core using NaI/I₂.

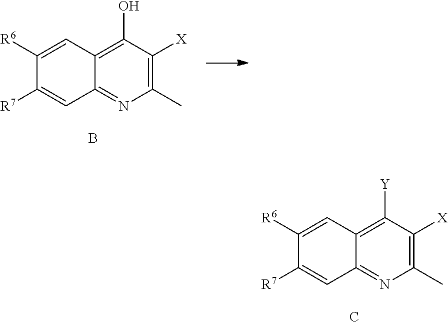

Phenol B is converted to aryl dihalide C under standard conditions. For example, conversion of the phenol to an aryl chloride may be accomplished with a standard chlorinating reagent known to those of skill in the art, such as, but not limited to POCl₃, PCl₅ or Ph₂POCl, preferably POCl₃, in the presence of an organic base, such as triethylamine or diisopropylethylamine.

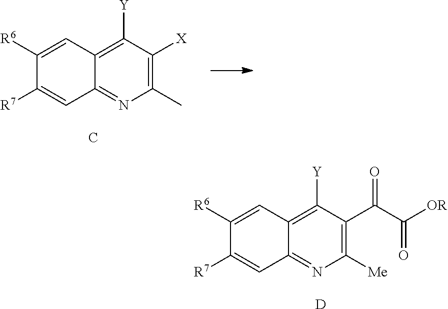

Aryl dihalide C is converted to ketone D by first chemoselective transformation of the 3-halo group to an aryl metal reagent, for example an aryl Grignard reagent, and then reaction of this intermediate with an activated carboxylic acid, for example methyl chlorooxoacetate. Those skilled in the art will recognize that other aryl metal reagents, such as, but not limited to, an aryl cuprate, aryl zinc, could be employed as the nucleophilic coupling partner. Those skilled in the art will also recognize that the electrophilic coupling partner could be also be replaced by another carboxylic acid derivative, such as a carboxylic ester, activated carboxylic ester, acid fluoride, acid bromide, Weinreb amide or other amide derivative.

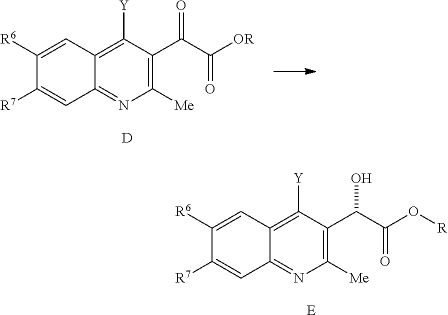

Ketone D is stereoselectively reduced to chiral alcohol E by any number of standard ketone reduction methods, such as rhodium catalyzed transfer hydrogenation using ligand Z (prepared analogously to the procedure in J. Org. Chem., 2002, 67(15), 5301-530, herein incorporated by reference),

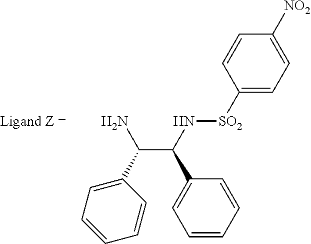 [SMILES: N[C@@H](c1ccccc1)[C@@H](NS(=O)(=O)c1ccc([N+](=O)[O-])cc1)c1ccccc1]

dichloro(pentamethylcyclopentadienyl)rhodium (III) dimer and formic acid as the hydrogen surrogate. Those skilled in the art will recognize that the hydrogen source could also be cyclohexene, cyclohexadiene, ammonium formate, isopropanol or that the reaction could be done under a hydrogen atmosphere. Those skilled in the art will also recognize that other transition metal catalysts or precatalysts could also be employed and that these could be composed of rhodium or other transition metals, such as, but not limited to, ruthenium, Iridium, palladium, platinum or nickel. Those skilled in the art will also recognize that the enantioselectivity in this reduction reaction could also be realized with other chiral phosphorous, sulfur, oxygen or nitrogen centered ligands, such as 1,2-diamines or 1,2-aminoalcohols of the general formula:

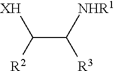

X═O, NR⁴
R¹=alkyl, aryl, benzyl, SO₂-alkyl, SO₂-aryl
R², R³═H, alkyl, aryl or R², R³ may link to form a cycle
R⁴═H, alkyl, aryl, alkyl-aryl
   wherein the alkyl and aryl groups may optionally be substituted with alkyl, nitro, haloalkyl, halo, NH₂, NH(alkyl), N(alkyl)₂, OH or —O-alkyl.

Preferred 1,2-diamines and 1,2-aminoalcohols are the following:

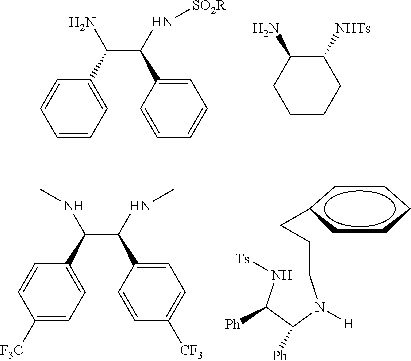

R=Me, p-toyl, o-nitrophenyl, p-nitrophenyl, 2,4,6-trimethylphenyl, 2,4,6-triisopropylphenyl, 2-naphthyl

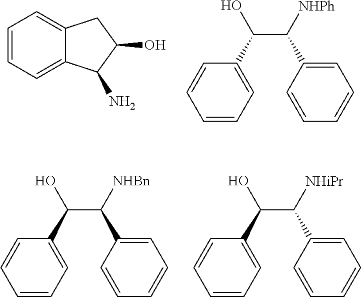

R may also be, for example, camphoryl, trifluoromethyl, alkylphenyl, nitrophenyl, halophenyl (F, Cl, Br, I), pentafluorophenyl, aminophenyl or alkoxyphenyl. Those skilled in the art will also recognize that this transformation may also be accomplished with hydride transfer reagents such as, but not limited to, the chiral CBS oxazaborolidine catalyst in combination with a hydride source such as, but not limited to, catechol borane.

Preferably the step of stereoselectively reducing ketone D to chiral alcohol E is achieved through the use of rhodium catalyzed transfer hydrogenation using ligand Z,

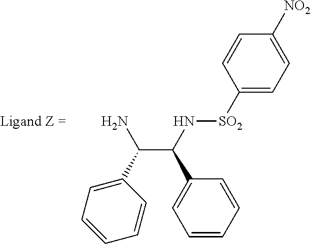 [SMILES: N[C@@H](c1ccccc1)[C@@H](NS(=O)(=O)c1ccc([N+](=O)[O-])cc1)c1ccccc1]

dichloro(pentamethylcyclopentadienyl)rhodium (III) dimer and formic acid as the hydrogen surrogate. These conditions allow for good enantiomeric excess, such as, for example greater than 98.5%, and a faster reaction rate. These conditions also allow for good catalyst loadings and efficient batch work-ups.

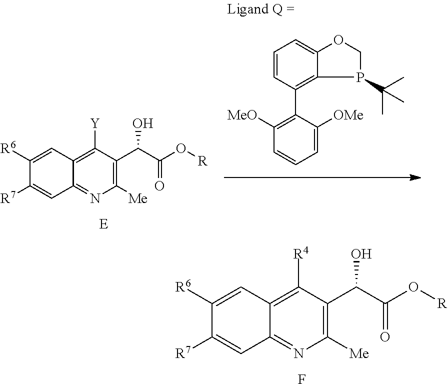

Aryl halide E is subjected to a diastereoselective Suzuki coupling reaction employing chiral phosphine ligand Q in combination with a palladium catalyst or precatalyst, preferably tris(dibenzylideneacetone)dipalladium(0) (Pd₂dba₃), a base and an appropriate boronic acid or boronate ester in an appropriate solvent mixture. Chiral phosphine ligand Q may be synthesized according to the procedure described in Angew. Chem. Int. Ed. 2010, 49, 5879-5883 and Org. Lett., 2011, 13, 1366-1369, the teachings of which are herein incorporated by reference.

While chiral phosphine Q is exemplified above, a person of skill in the art would recognize that other biaryl monophosphorus ligands described in Angew. Chem. Int. Ed. 2010, 49, 5879-5883; Org. Lett., 2011, 13, 1366-1369, and in pending PCT/US2002/030681 the teachings of which are each hereby incorporated by reference, could be used in the diastereoselective Suzuki coupling reaction.

Suitable biaryl monophosphorus ligands for use in the diastereoselective Suzuki coupling reaction are shown below:

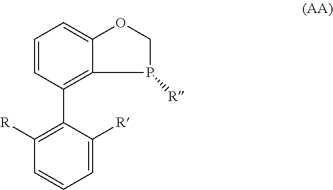

wherein R═R′═H; R″=tert-butyl; or R═OMe; R′═H; R″=tert-butyl; or R═N(Me)₂; R′═H; R″=tert-butyl.

A person of skill in the art will recognize that the particular boronic acid or boronate ester will depend upon the desired R⁴ in the final inhibitor H. Selected examples of the boronic acid or boronate ester that may be used are, for example:

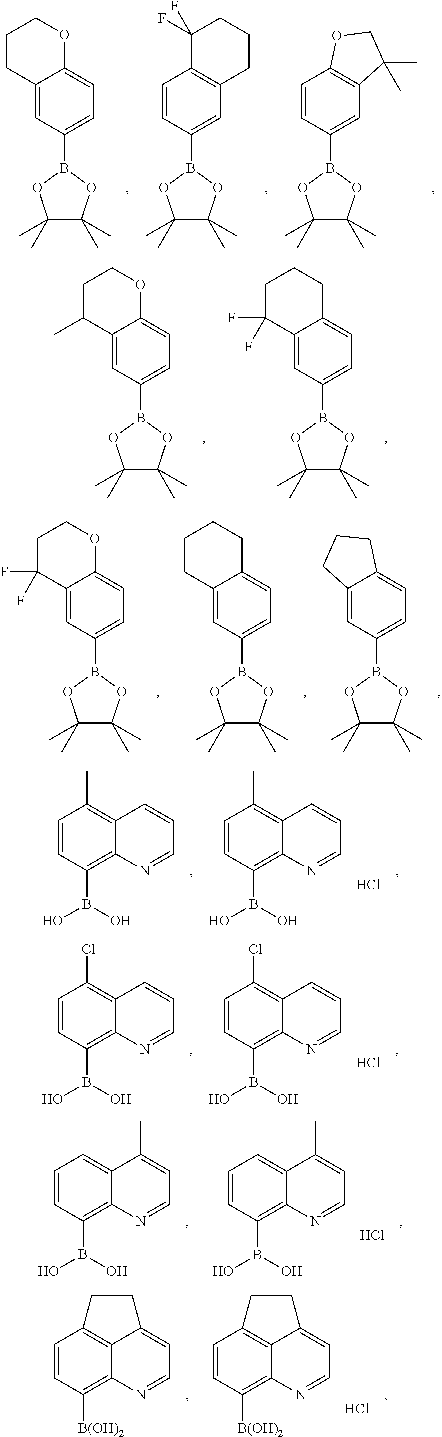

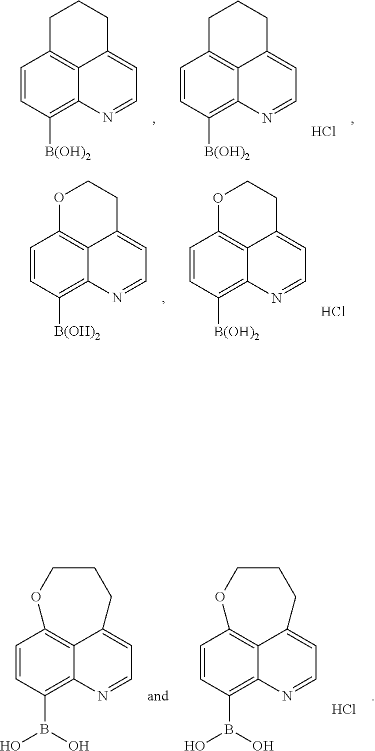

This cross-coupling reaction step provides conditions whereby the use of a chiral phosphine Q provides excellent conversion and good selectivity, such as, for example, 5:1 to 6:1, in favor of the desired atropisomer in the cross-coupling reaction.

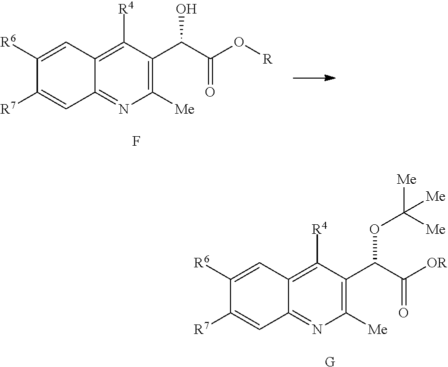

Chiral alcohol F is converted to tert-butyl ether G under BrØnstead- or Lewis-acid catalysis with a source tert-butyl cation or its equivalent. The catalyst may be, for example, Zn(SbF₆) or AgSbF₆ or trifluoromethanesulfonimide. Preferably, the catalyst is trifluoromethanesulfonimide which increases the efficiency of the reagent t-butyl-trichloroacetimidate. In addition, this catalyst allows the process to be scaled.

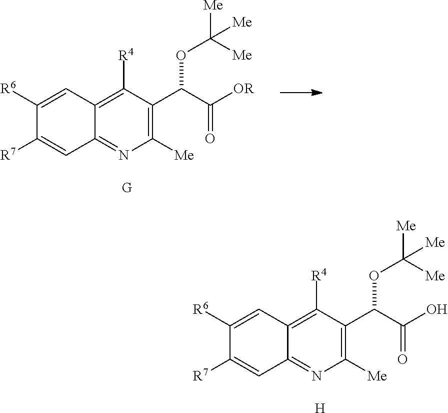

Ester G is converted to the final inhibitor H through a standard saponification reaction in a suitable solvent mixture. Inhibitor H may optionally be converted to a salt thereof using standard methods.

### IV. General Scheme IA—General Multi-Step Synthetic Method to Prepare Compound 1001 or a Salt Thereof

In one embodiment, the present invention is directed to a general multi-step synthetic method for preparing Compound 1001 or salt thereof:

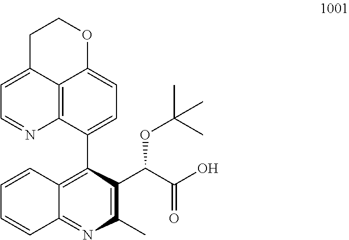

according to the following General Scheme IA:

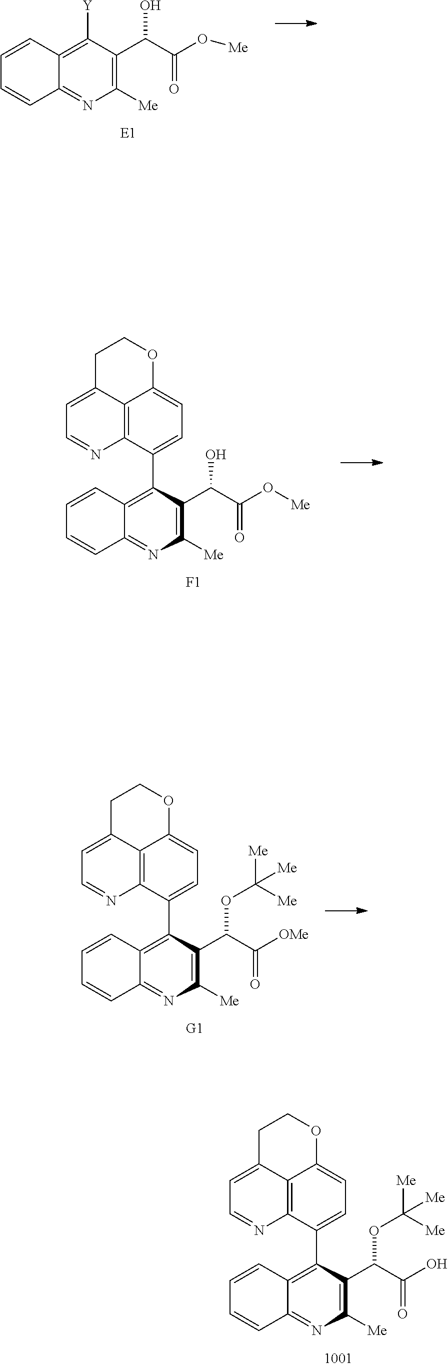

wherein Y is I, Br or Cl;
wherein the process comprises:
coupling aryl halide E1 under diastereoselective Suzuki coupling conditions in the presence of a chiral biaryl monophosphorus ligand having Formula (AA):

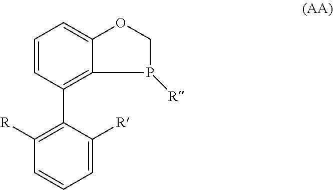

wherein R═R′═H; R″=tert-butyl; or R═OMe; R′═H; R″=tert-butyl; or R═N(Me)₂; R′═H; R″=tert-butyl;
   in combination with a palladium catalyst or precatalyst, and a base and a boronic acid or boronate ester in a solvent mixture;
converting chiral alcohol FI to tert-butyl ether G1 under BrØnstead- or Lewis-acid catalysis with a source tert-butyl cation or its equivalent;
saponifying ester G1 to Compound 1001 in a solvent mixture; and
optionally converting Compound 1001 to a salt.

In one embodiment, the present invention is directed to a general multi-step synthetic method for preparing Compound 1001 or salt thereof:

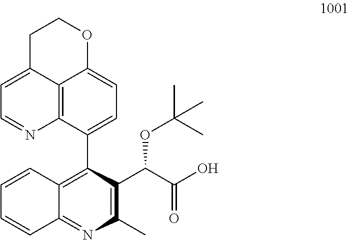

according to the following General Scheme IA:

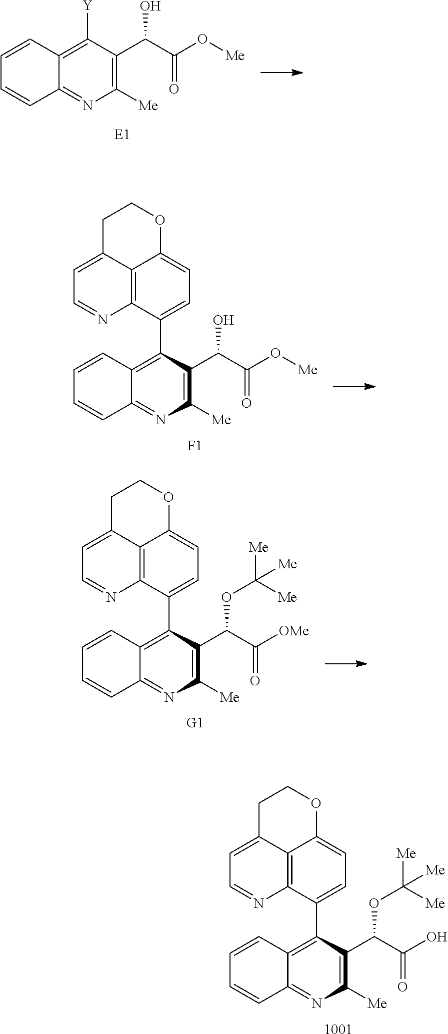

wherein Y is I, Br or Cl;
wherein the process comprises:
subjecting aryl halide E1 to a diastereoselective Suzuki coupling reaction employing a chiral biaryl monophosphorus ligand of Formula (AA):

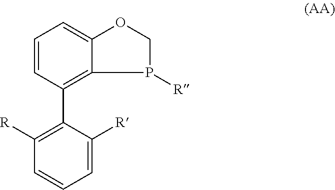

R═R′═H; R″=tert-butyl; or R═OMe; R′═H; R″=tert-butyl; or R═N(Me)₂; R′═H; R″=tert-butyl;
   in combination with a palladium catalyst or precatalyst, a base and an appropriate boronic acid or boronate ester in an appropriate solvent mixture;
converting chiral alcohol F1 to tert-butyl ether G1 under BrØnstead- or Lewis-acid catalysis with a source tert-butyl cation or its equivalent;
converting ester G1 to Compound 1001 through a standard saponification reaction in a suitable solvent mixture; and
optionally converting Compound 1001 to a salt thereof using standard methods.

The boronic acid or boronate ester may be selected from, for example:

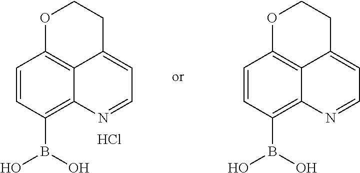 [SMILES: OB(O)c1ccc2c3c(ccnc13)CCO2 ; Cl ; OB(O)c1ccc2c3c(ccnc13)CCO2]

Preferably, the boronic acid or boronate ester is:

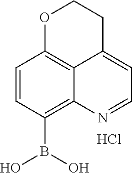 [SMILES: OB(O)c1ccc2c3c(ccnc13)CCO2 ; Cl]

### V. General Scheme IIA—General Multi-Step Synthetic Method to Prepare Compound 1001 or a Salt Thereof

In one embodiment, the present invention is directed to a general multi-step synthetic method for preparing a Compound 1001 or salt thereof:

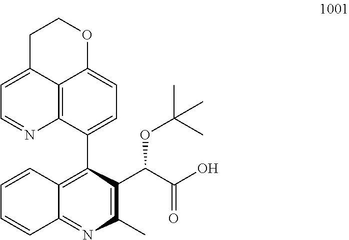

according to the following General Scheme IIA:

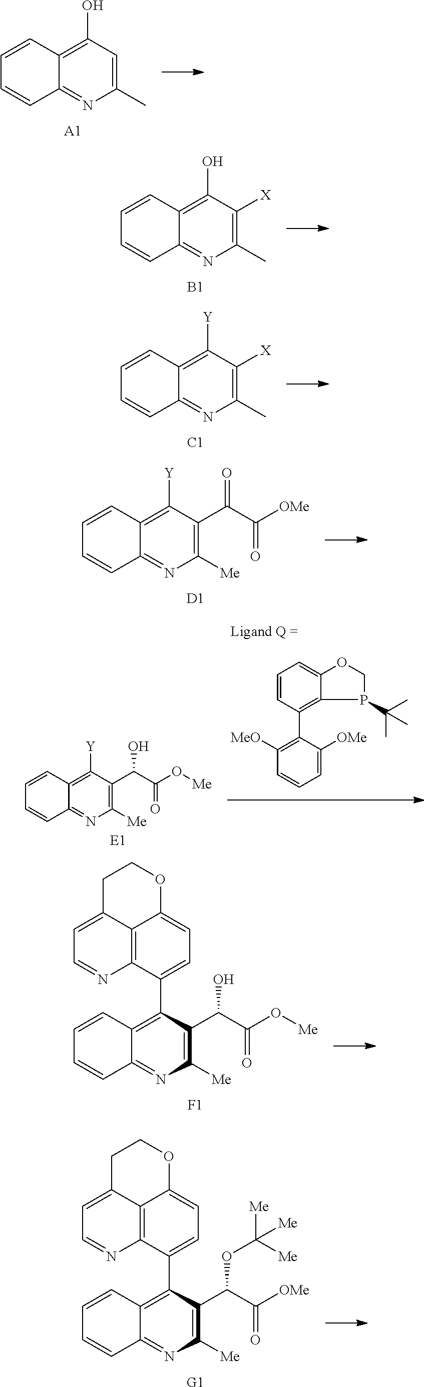

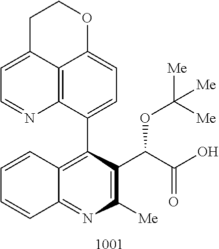

wherein:
X is I or Br; and
Y is Cl when X is Br or I, or Y is Br when X is I, or Y is I;
   wherein the process comprises:
converting 4-hydroxyquinoline A1 to phenol B1 via a regioselective halogenation reaction at the 3-position of the quinoline core;
converting phenol B1 to aryl dihalide C1 through activation of the phenol with an activating reagent and subsequent treatment with a halide source in the presence of an organic base;
converting aryl dihalide C1 to ketone D1 by chemoselectively transforming the 3-halo group to an aryl metal reagent and then reacting the aryl metal reagent with an activated carboxylic acid;
stereoselectively reducing ketone D1 to chiral alcohol E1 by asymmetric ketone reduction methods;
diastereoselectively coupling aryl halide E1 under Suzuki coupling reaction conditions in the presence of a chiral phosphine ligand Q in combination with a palladium catalyst or precatalyst, a base and a boronic acid or boronate ester in a solvent mixture;
converting chiral alcohol F1 to tert-butyl ether G1 under BrØnstead- or Lewis-acid catalysis with a source tert-butyl cation or its equivalent;
saponifying ester G1 to Compound 1001 in a solvent mixture; and
optionally converting Compound 1001 to a salt thereof.

In one embodiment, the present invention is directed to a general multi-step synthetic method for preparing a Compound 1001 or salt thereof:

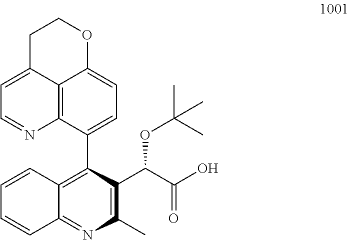

according to the following General Scheme IIA:

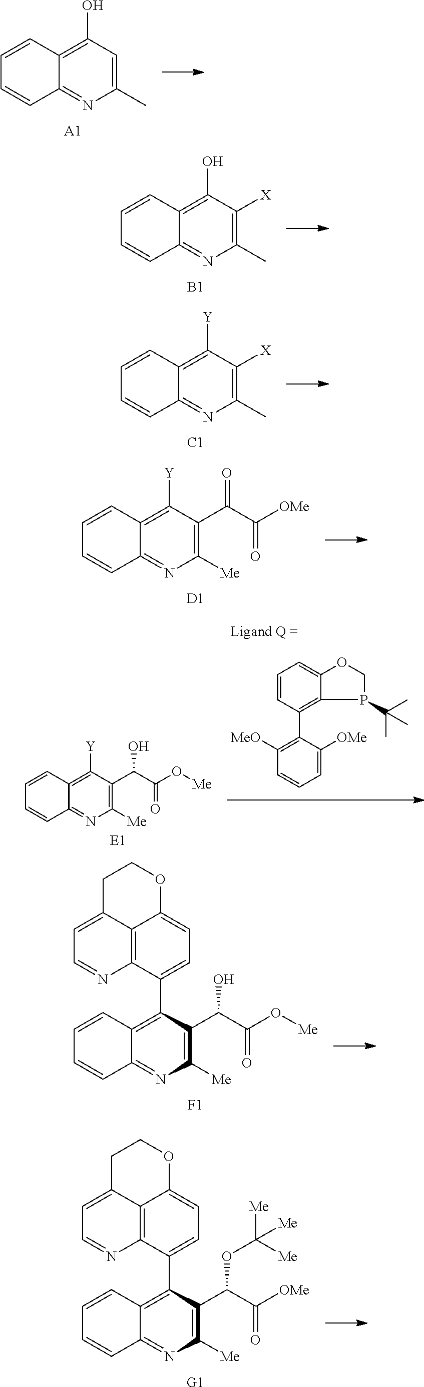

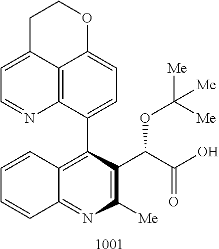

wherein:
X is I or Br, and
Y is Cl when X is Br or I, or Y is Br when X is I, or Y is I;
   wherein the process comprises:
converting 4-hydroxyquinoline A1 to phenol B1 via a regioselective halogenation reaction at the 3-position of the quinoline core;
converting phenol B1 to aryl dihalide C1 through activation of the phenol with a suitable activating reagent and subsequent treatment with an appropriate halide source in the presence of an organic base;
converting aryl dihalide C1 to ketone D1 by first chemoselective transformation of the 3-halo group to an aryl metal reagent, and then reaction of this intermediate with an activated carboxylic acid;
stereoselectively reducing ketone D1 to chiral alcohol E1 by standard asymmetric ketone reduction methods;
subjecting aryl halide E1 to a diastereoselective Suzuki coupling reaction employing chiral phosphine Q in combination with a palladium catalyst or precatalyst, a base and an appropriate boronic acid or boronate ester in an appropriate solvent mixture;
converting chiral alcohol F1 to tert-butyl ether G1 under BrØnstead- or Lewis-acid catalysis with a source tert-butyl cation or its equivalent;
converting ester G1 to Compound 1001 through a standard saponification reaction in a suitable solvent mixture; and
optionally converting Compound 1001 to a salt thereof using standard methods.

The boronic acid or boronate ester may be selected from, for example:

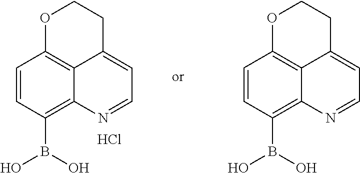 [SMILES: OB(O)c1ccc2c3c(ccnc13)CCO2 ; Cl ; OB(O)c1ccc2c3c(ccnc13)CCO2]

Preferably, the boronic acid or boronate ester is:

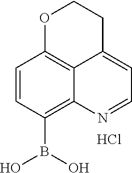 [SMILES: OB(O)c1ccc2c3c(ccnc13)CCO2 ; Cl]

### VI. General Schemes IA and IIA—Individual Steps of the Synthetic Method to Prepare Compound 1001, or a Salt Thereof

Additional embodiments of the invention are directed to the individual steps of the multistep general synthetic method described above in Sections IV and V above, namely General Schemes IA and IIA, and the individual intermediates used in these steps. These individual steps and intermediates of the present invention are described in detail below. All substituent groups in the steps described below are as defined in the multi-step method above.

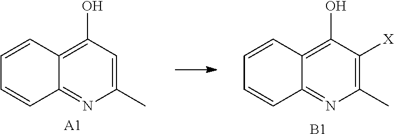

Readily or commercially available 4-hydroxyquinoline A1 is converted to phenol B1 via a regioselective halogenation reaction at the 3-position of the quinoline core. This may be accomplished with electrophilic halogenation reagents known to those of skill in the art, such as, for example, but not limited to NIS, NBS, I₂, NaI/I₂, Br₂, Br-i, Cl—I or Br₃pyr. Preferably, 4-hydroxyquinoline A1 is converted to phenol B1 via a regioselective iodination reaction at the 3-position of the quinoline core. More preferably, 4-hydroxyquinoline A1 is converted to phenol B1 via a regioselective iodination reaction at the 3-position of the quinoline core using NaI/I₂.

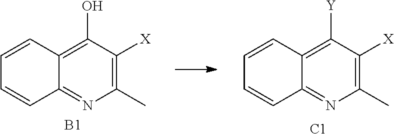

Phenol B1 is converted to aryl dihalide C1 under standard conditions. For example, conversion of the phenol to an aryl chloride may be accomplished with a standard chlorinating reagent known to those of skill in the art, such as, but not limited to POCl₃, PCl₅ or Ph₂POCl, preferably POCl₃, in the presence of an organic base, such as triethylamine or diisopropylethylamine.

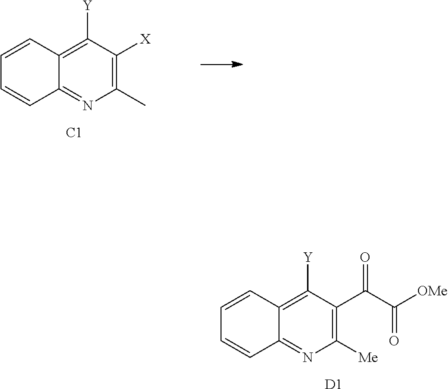

Aryl dihalide C1 is converted to ketone D1 by first chemoselective transformation of the 3-halo group to an aryl metal reagent, for example an aryl Grignard reagent, and then reaction of this intermediate with an activated carboxylic acid, for example methyl chlorooxoacetate. Those skilled in the art will recognize that other aryl metal reagents, such as, but not limited to, an aryl cuprate, aryl zinc, could be employed as the nucleophilic coupling partner. Those skilled in the art will also recognize that the electrophilic coupling partner could be also be replaced by another carboxylic acid derivative, such as a carboxylic ester, activated carboxylic ester, acid fluoride, acid bromide, Weinreb amide or other amide derivative.

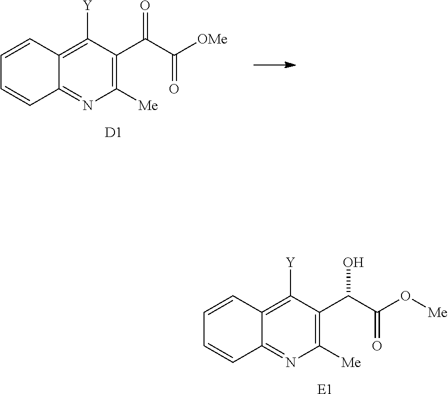

Ketone D1 is stereoselectively reduced to chiral alcohol E1 by any number of standard ketone reduction methods, such as rhodium catalyzed transfer hydrogenation using ligand Z (prepared analogously to the procedure in J. Org. Chem., 2002, 67(15), 5301-530, herein incorporated by reference),

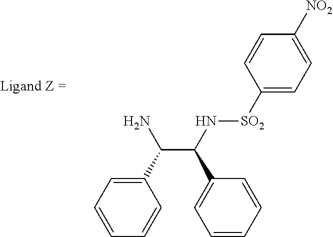 [SMILES: N[C@@H](c1ccccc1)[C@@H](NS(=O)(=O)c1ccc([N+](=O)[O-])cc1)c1ccccc1]

dichloro(pentamethylcyclopentadienyl)rhodium (III) dimer and formic acid as the hydrogen surrogate. Those skilled in the art will recognize that the hydrogen source could also be cyclohexene, cyclohexadiene, ammonium formate, Isopropanol or that the reaction could be done under a hydrogen atmosphere. Those skilled in the art will also recognize that other transition metal catalysts or precatalysts could also be employed and that these could be composed of rhodium or other transition metals, such as, but not limited to, ruthenium, iridium, palladium, platinum or nickel. Those skilled in the art will also recognize that the enantioselectivity in this reduction reaction could also be realized with other chiral phosphorous, sulfur, oxygen or nitrogen centered ligands, such as 1,2-diamines or 1,2-aminoalcohols of the general formula:

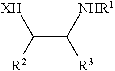

X═O, NR⁴
R¹=alkyl, aryl, benzyl, SO₂-alkyl, SO₂-aryl
R², R³═H, alkyl, aryl or R², R³ may link to form a cycle
R⁴═H, alkyl, aryl, alkyl-aryl
   wherein the alkyl and aryl groups may optionally be substituted with alkyl, nitro, haloalkyl, halo, NH₂, NH(alkyl), N(alkyl)₂, OH or —O-alkyl.

Preferred 1,2-diamines or 1,2-aminoalcohols include the following structures:

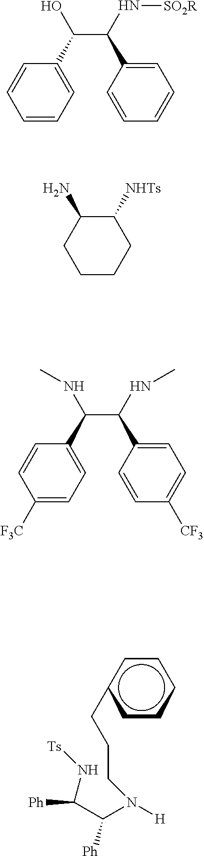

R-Me, p-tolyl, o-nitrophenyl, p-nitrophenyl, 2,4,6-trimethylphenyl, 2,4,6-triisopropylphenyl, 2-naphthyl

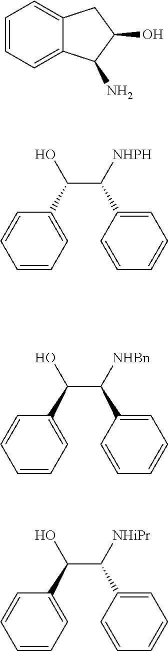

R may also be, for example, camphoryl, trifluoromethyl, alkylphenyl, nitrophenyl, halophenyl (F, Cl, Br, I), pentafluorophenyl, aminophenyl or alkoxyphenyl. Those skilled in the art will also recognize that this transformation may also be accomplished with hydride transfer reagents such as, but not limited to, the chiral CBS oxazaborolidine catalyst in combination with a hydride source such as, but not limited to, catechol borane.

Preferably the step of stereoselectively reducing ketone D1 to chiral alcohol E1I is achieved through the use of rhodium catalyzed transfer hydrogenation using ligand Z,

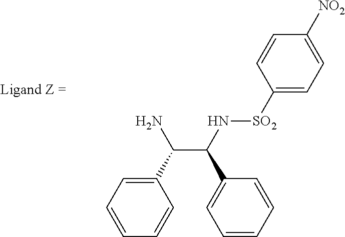 [SMILES: N[C@@H](c1ccccc1)[C@@H](NS(=O)(=O)c1ccc([N+](=O)[O-])cc1)c1ccccc1]

dichloro(pentamethylcyclopentadienyl)rhodium (III) dimer and formic acid as the hydrogen surrogate. These conditions allow for good enantiomeric excess, such as, for example greater than 98.5%, and a faster reaction rate. These conditions also allow for good catalyst loadings and efficient batch work-ups.

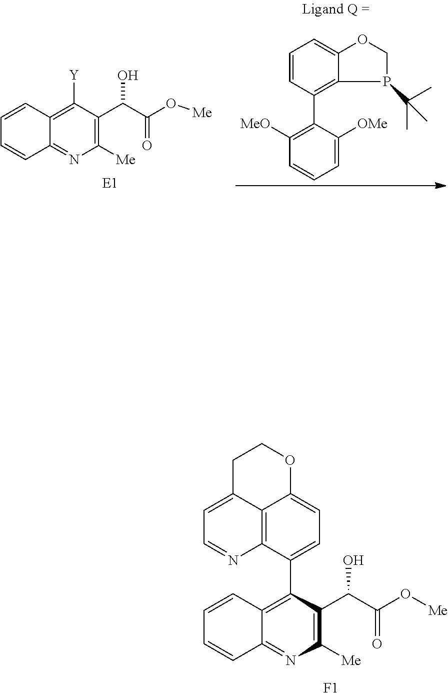

Aryl halide E1 is subjected to a diastereoselective Suzuki coupling reaction employing chiral phosphine Q (synthesized according to the procedure described in Angew. Chem. Int. Ed. 2010, 49, 5879-5883 and Org. Lett., 2011, 13, 1366-1369, herein incorporated by reference) in combination with a palladium catalyst or precatalyst, preferably Pd₂dba₃, a base and an appropriate boronic acid or boronate ester in an appropriate solvent mixture. While chiral phosphine Q is exemplified above, a person of skill in the art would recognize that other biaryl monophosphorus ligands described in Angew. Chem. Int. Ed. 2010, 49, 5879-5883 and Org. Lett., 2011, 13, 1366-1369, and in pending PCT/US2002/030681 could be used in the diastereoselective Suzuki coupling reaction. Suitable biaryl monophosphorus ligands for use in the diastereoselective Suzuki coupling reaction are shown below having Formula (AA):

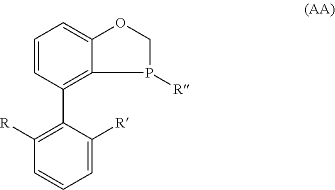

wherein R═R′═H; R″=tert-butyl; or R═OMe; R′═H; R″=tert-butyl; or R═N(Me)₂; R′═H; R″=tert-butyl.

The boronic acid or boronate ester may be selected from, for example:

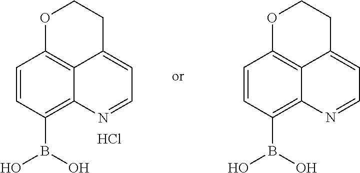 [SMILES: OB(O)c1ccc2c3c(ccnc13)CCO2 ; Cl ; OB(O)c1ccc2c3c(ccnc13)CCO2]

Preferably, the boronic acid or boronate ester is:

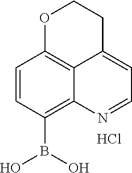 [SMILES: OB(O)c1ccc2c3c(ccnc13)CCO2 ; Cl]

This cross-coupling reaction step provides conditions whereby the use of a chiral phosphine Q provides excellent conversion and good selectivity, such as, for example, 5:1 to 6:1, in favor of the desired atropisomer in the cross-coupling reaction.

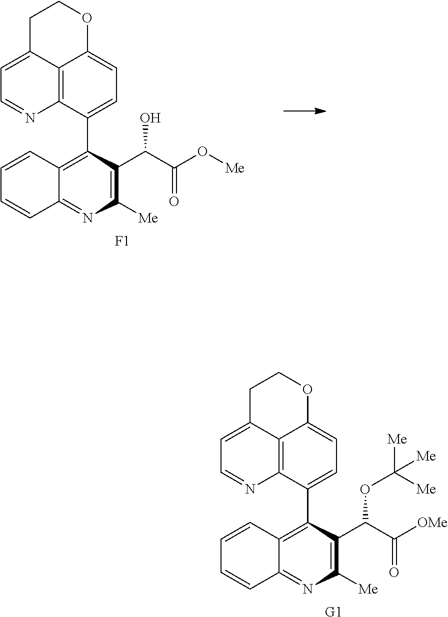

Chiral alcohol F1 is converted to tert-butyl ether G1 under BrØnstead- or Lewis-acid catalysis with a source tert-butyl cation or its equivalent. The catalyst may be, for example, Zn(SbF₆) or AgSbF₆ or trifluoromethanesulfonimide. Preferably, the catalyst is trifluoromethanesulfonimide which increases the efficiency of the reagent t-butyl-trichloroacetimidate. In addition, this catalyst allows the process to be scaled.

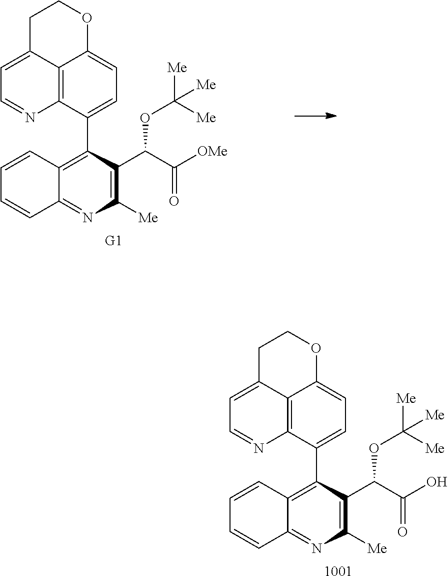

Ester G1 is converted to Compound 1001 through a standard saponification reaction in a suitable solvent mixture. Inhibitor H may optionally be converted to a salt thereof using standard methods.

### VII. General Scheme III—General Method to Prepare a Quinoline-8-Boronic Acid Derivative or a Salt Thereof

In one embodiment, the present invention is directed to a general multi-step synthetic method for preparing a quinoline-8-boronic acid derivative or a salt thereof, according to the following General Scheme III:

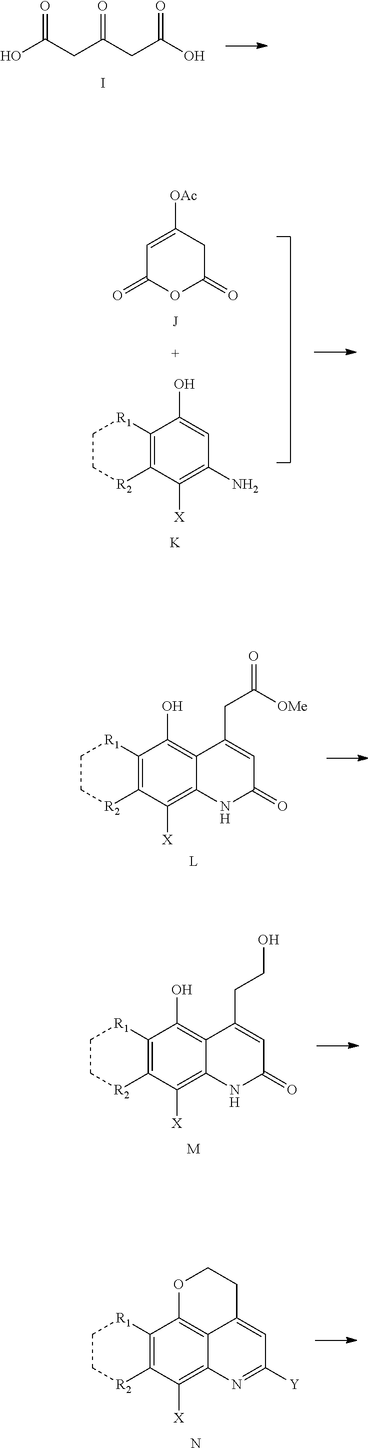

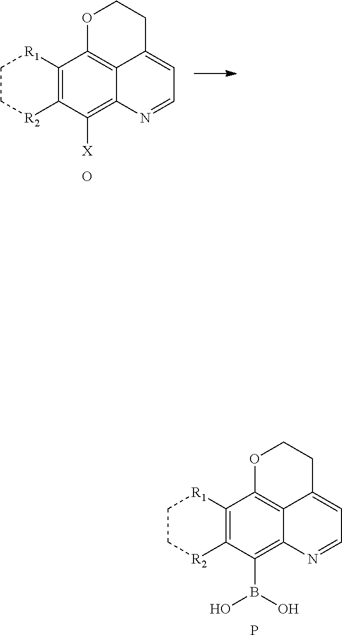

wherein:
X is Br or I;
Y is Br or Cl; and
R₁ and R₂ may either be absent or linked to form a cycle; preferably R₁ and R₂ are absent.

Diacid I is converted to cyclic anhydride J under standard conditions. Anhydride J is then condensed with meta-aminophenol K to give quinolone L. The ester of compound L is then reduced under standard conditions to give alcohol M, which then undergoes a cyclization reaction to give tricyclic quinoline N via activation of the alcohol as its corresponding alkyl chloride. Those skilled in the art will recognize that a number of different activation/cyclicaztion conditions can be envisaged to give compound N where Y═Cl, including, but not limited to (COCl)₂, SOCl₂ and preferably POCl₃. Alternatively, the alcohol could also be activated as the alkyl bromide under similar activation/cyclization conditions, including, but not limited to POBr₃ and PBr₅ to give tricyclic quinoline N, where Y═Br. Reductive removal of halide Y is then achieved under acidic conditions with a reductant such as, but not limited to, Zinc metal, to give compound O. Finally, halide X in compound O dissolved in a suitable solvent, such as toluene, is converted to the corresponding boronic acid P, sequentially via the corresponding intermediate aryl lithium reagent and boronate ester. Those skilled in the art will recognize that this could be accomplished by controlled halogen/lithium exchange with an alkyllithium reagent, followed by quenching with a trialkylborate reagent. Those skilled in the art will also recognize that this could be accomplished through a transition metal catalyzed cross coupling reaction between compound O and a diborane species, followed by a hydrolysis step to give compound P. Compound P may optionally be converted to a salt thereof using standard methods.

The following examples are provided for purposes of illustration, not limitation.

### EXAMPLES

In order for this invention to be more fully understood, the following examples are set forth. These examples are for the purpose of illustrating embodiments of this invention, and are not to be construed as limiting the scope of the invention in any way. The reactants used in the examples below may be obtained either as described herein, or if not described herein, are themselves either commercially available or may be prepared from commercially available materials by methods known in the art. Certain starting materials, for example, may be obtained by methods described in the International Patent Applications WO 2007/131350 and WO 2009/062285.

Unless otherwise specified, solvents, temperatures, pressures, and other reaction conditions may be readily selected by one of ordinary skill in the art. Typically, reaction progress may be monitored by High Pressure Liquid Chromatography (HPLC), if desired, and intermediates and products may be purified by chromatography on silica gel and/or by recrystallization.

In one embodiment, the present invention is directed to the multi-step synthetic method for preparing Compound 1001 as set forth in Examples 1-13. In another embodiment, the invention is directed to each of the individual steps of Examples 1-13 and any combination of two or more successive steps of Examples 1-13.

Abbreviations or symbols used herein include: Ac: acetyl; AcOH: acetic acid; Ac₂O: acetic anhydride; Bn: benzyl; Bu: butyl; DMAc: N,N-Dimethylacetamide; Eq: equivalent; Et: ethyl; EtOAc: ethyl acetate; EtOH: ethanol: HPLC: high performance liquid chromatography; IPA: isopropyl alcohol; ⁱPr or i-Pr: 1-methylethyl (iso-propyl); KF: Karl Fischer; LOD: limit of detection; Me: methyl; MeCN: acetonitrile; MeOH: methanol; MS: mass spectrometry (ES: electrospray); MTBE: methyl-t-butyl ether; BuLi: n-butyl lithium; NMR: nuclear magnetic resonance spectroscopy; Ph: phenyl; Pr: propyl; tert-butyl or t-butyl: 1,1-dimethylethyl; TFA: trifluoroacetic acid; and THF: tetrahydrofuran.

### Example 1

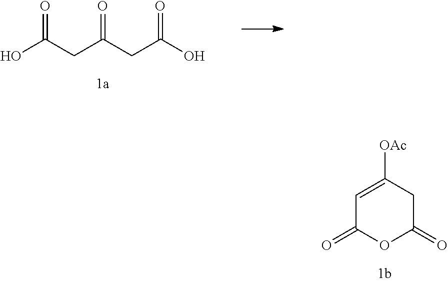 [SMILES: O=C(O)CC(=O)CC(=O)O ; CC(=O)OC1=CC(=O)OC(=O)C1]

1a (600 g, 4.1 mol) was charged into a dry reactor under nitrogen followed by addition of Ac₂O (1257.5 g, 12.3 mol, 3 eq.). The resulting mixture was heated at 40° C. at least for 2 hours. The batch was then cooled to 30° C. over 30 minutes. A suspension of 1b in toluene was added to seed the batch if no solid was observed. After toluene (600 mL) was added over 30 minutes, the batch was cooled to −5˜−10° C. and was held at this temperature for at least 30 minutes. The solid was collected by filtration under nitrogen and rinsed with heptanes (1200 mL). After being dried under vacuum at room temperature, the solid was stored under nitrogen at least below 20° C. The product 1b was obtained with 77% yield. ¹H NMR (500 MHz, CDCl₃): δ=6.36 (s, 1H), 3.68 (s, 2H), 2.30 (s, 3H).

### Example 2

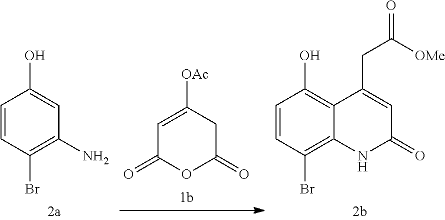

2a (100 g, 531 mmol) and 1b (95 g, 558 mmol) were charged into a clean and dry reactor under nitrogen followed by addition of fluorobenzene (1000 mL). After being heated at 35-37° C. for 4 hours, the batch was cooled to 23° C. Concentrated H₂SO₄ (260.82 g, 2659.3 mmol, 5 eq.) was added while maintaining the batch temperature below 35° C. The batch was first heated at 30-35° C. for 30 minutes and then at 40-45° C. for 2 hours. 4-Methyl morpholine (215.19 g, 2127 mmol, 4 eq.) was added to the batch while maintaining the temperature below 50° C. Then the batch was agitated for 30 minutes at 40-50° C. MeOH (100 mL) was then added while maintaining the temperature below 55° C. After the batch was held at 50-55° C. for 2 hours, another portion of MeOH (100 mL) was added. The batch was agitated for another 2 hours at 50-55° C. After fluorobenzene was distilled to a minimum amount, water (1000 mL) was added. Further distillation was performed to remove any remaining fluorobenzene. After the batch was cooled to 30° C., the solid was collected by filtration with cloth and rinsed with water (400 mL) and heptane (200 mL). The solid was dried under vacuum below 50° C. to reach KF<0.1%. Typically, the product 2b was obtained in 90% yield with 98 wt %. ¹H NMR (500 MHz, DMSO-d₆): δ=10.83 (s, 1H), 9.85 (s, bs, 1H), 7.6 (d, 1H, J=8.7 Hz), 6.55 (d, 1H, J=8.7 Hz), 6.40 (s, 1H), 4.00 (s, 2H), 3.61 (s, 3H).

### Example 3

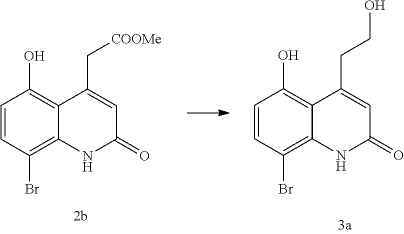 [SMILES: COC(=O)Cc1cc(=O)[nH]c2c(Br)ccc(O)c12 ; O=c1cc(CCO)c2c(O)ccc(Br)c2[nH]1]

2b (20 g, 64 mmol) was charged into a clean and dry reactor followed by addition of THF (140 mL). After the resulting mixture was cooled to 0° C., Vitride® (Red-AI, 47.84 g, 65 wt %, 154 mmol) in toluene was added while maintaining an internal temperature at 0-5° C. After the batch was agitated at 5-10° C. for 4 hours, IPA (9.24 g, 153.8 mmol) was added while maintaining the temperature below 10° C. Then the batch was agitated at least for 30 minutes below 25° C. A solution of HCl in IPA (84.73 g, 5.5 M, 512 mmol) was added into the reactor while maintaining the temperature below 40° C. After about 160 mL of the solvent was distilled under vacuum below 40° C., the batch was cooled to 20-25° C. and then aqueous 6M HCl (60 mL) was added while maintaining the temperature below 40° C. The batch was cooled to 25° C. and agitated for at least 30 minutes. The solid was collected by filtration, washed with 40 mL of IPA and water (1V/1V), 40 mL of water and 40 mL of heptanes. The solid was dried below 60° C. in a vacuum oven to reach KF<0.5%. Typically, the product 3a was obtained in 90-95% yield with 95 wt %. ¹H NMR (400 MHz, DMSO-d₆): δ=10.7 (s, 1H), 9.68 (s, 1H), 7.59 (d, 1H, J=8.7 Hz), 6.64 (, 1H, J=8.7 Hz), 6.27 (s, 1H), 4.62 (bs, 1H), 3.69 (t, 2H, J=6.3 Hz), 3.21 (t, 2H, J=6.3 Hz).

### Example 4

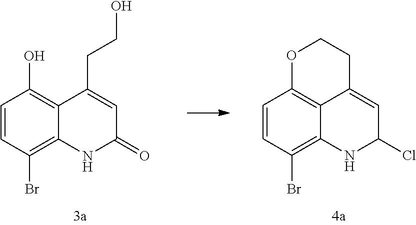 [SMILES: O=c1cc(CCO)c2c(O)ccc(Br)c2[nH]1 ; ClC1C=C2CCOc3ccc(Br)c(c32)N1]

3a (50 g, 174.756 mmol) and acetonitrile (200 mL) were charged into a dry and clean reactor. After the resulting mixture was heated to 65° C., POCl₃ (107.18 g, 699 mmol, 4 eq.) was added while maintaining the internal temperature below 75° C. The batch was then heated at 70-75° C. for 5-6 hours. The batch was cooled to 20° C. Water (400 mL) was added at least over 30 minutes while maintaining the internal temperature below 50° C. After the batch was cooled to 20-25° C. over 30 minutes, the solid was collected by filtration and washed with water (100 mL). The wet cake was charged back into the reactor followed by addition of 1M NaOH (150 mL). After the batch was agitated at least for 30 minutes at 25-35° C., it was verified that the pH was greater than 12. Otherwise, more 6M NaOH was needed to adjust the pH>12. After the batch was agitated for 30 minutes at 25-35° C., the solid was collected by filtration, washed with water (200 mL) and heptanes (200 mL). The solid was dried in a vacuum oven below 50° C. to reach KF<2%. Typically, the product 4a was obtained at about 75-80% yield. ¹H NMR (400 MHz, CDCl₃): δ=7.90 (d, 1H, J=8.4 Hz), 7.16 (s, 1H), 6.89 (d, 1H, J=8.4 Hz), 4.44 (t, 2H, J=5.9 Hz), 3.23 (t, 2H, J=5.9 Hz). ¹³C NMR (100 MHz, CDCl₃): δ=152.9, 151.9, 144.9, 144.1, 134.6, 119.1, 117.0, 113.3, 111.9, 65.6, 28.3.

### Example 5

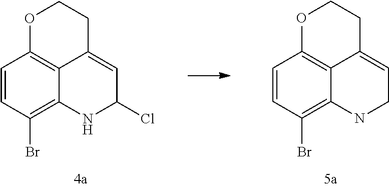 [SMILES: ClC1C=C2CCOc3ccc(Br)c(c32)N1 ; Brc1ccc2c3c1NCC=C3CCO2]

Zn powder (54 g, 825 mmol, 2.5 eq.) and TFA (100 mL) were charged into a dry and clean reactor. The resulting mixture was heated to 60-65° C. A suspension of 4a (100 g, 330 mmol) in 150 mL of TFA was added to the reactor while maintaining the temperature below 70° C. The charge line was rinsed with TFA (50 mL) into the reactor. After 1 hour at 65±5° C., the batch was cooled to 25-30° C. Zn powder was filtered off by passing the batch through a Celite pad and washing with methanol (200 mL). About 400 mL of solvent was distilled off under vacuum. After the batch was cooled to 20-25° C., 20% NaOAc (ca. 300 mL) was added at least over 30 minutes to reach pH 5-6. The solid was collected by filtration, washed with water (200 mL) and heptane (200 mL), and dried under vacuum below 45° C. to reach KF≦2%. The solid was charged into a dry reactor followed by addition of loose carbon (10 wt %) and toluene (1000 mL). The batch was heated at least for 30 minutes at 45-50° C. The carbon was filtered off above 35° C. and rinsed with toluene (200 mL). The filtrate was charged into a clean and dry reactor. After about 1000 mL of toluene was distilled off under vacuum below 50° C., 1000 mL of heptane was added over 30 minutes at 40-50° C. Then the batch was cooled to 0±5° C. over 30 minutes. After 30 minutes, the solid was collected and rinsed with 200 mL of heptane. The solid was dried under vacuum below 45° C. to reach KF≦500 ppm. Typically, the product 5a was obtained in about 90-95% yield. ¹H NMR (400 MHz, CDCl₃): δ=8.93 (m, 1H), 7.91 (dd, 1H, J=1.5, 8 Hz), 7.17 (m 1H), 6.90 (dd, 1H, J=1.6, 8.0 Hz), 4.46-4.43 (m, 2H), 3.28-3.23 (m, 2H). ¹³C NMR (100 MHz, CDCl₃): δ=152.8, 151.2, 145.1, 141.0, 133.3, 118.5, 118.2, 114.5, 111.1, 65.8, 28.4.

### Example 6

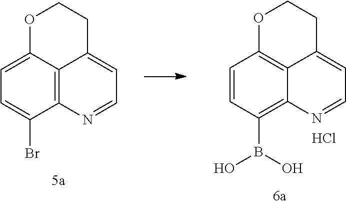 [SMILES: Brc1ccc2c3c(ccnc13)CCO2 ; OB(O)c1ccc2c3c(ccnc13)CCO2 ; Cl]

5a (1.04 kg, 4.16 mol) and toluene (8 L) were charged into the reactor. The batch was agitated and cooled to −50 to −55° C. BuLi solution (2.5 M in hexanes, 1.69 L, 4.23 mol) was charged slowly while maintaining the internal temperature between −45 to −50° C. The batch was agitated at −45° C. for 1 hour after addition. A solution of triisopropyl borate (0.85 kg, 4.5 mol) in MTBE (1.48 kg) was charged. The batch was warmed to 10° C. over 30 minutes. A solution of 5 N HCl in IPA (1.54 L) was charged slowly at 10° C., and the batch was warmed to 20° C. and stirred for 30 minutes. It was seeded with 6a crystal (10 g). A solution of aqueous concentrated HCl (0.16 L) in IPA (0.16 L) was charged slowly at 20° C. in three portions at 20 minute intervals, and the batch was agitated for 1 hour at 20° C. The solid was collected by filtration, rinsed with MTBE (1 kg), and dried to provide 6a (943 g, 88.7% purity, 80% yield). ¹H NMR (400 MHz, D₂0): δ 8.84 (d, 1H, J=4 Hz), 8.10 (m, 1H), 7.68 (d, 1H, J=6 Hz), 7.09 (m, 1H), 4.52 (m, 2H), 3.47 (m, 2H).

### Example 7

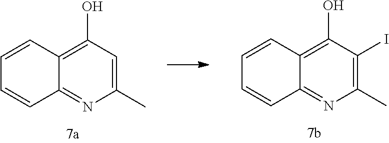

Iodine stock solution was prepared by mixing iodine (57.4 g, 0.23 mol) and sodium iodide (73.4 g, 0.49 mol) in water (270 mL). Sodium hydroxide (28.6 g, 0.715 mol) was charged into 220 mL of water. 4-Hydroxy-2 methylquinoline 7a (30 g, 0.19 mol) was charged, followed by acetonitrile (250 mL). The mixture was cooled to 10° C. with agitation. The above iodine stock solution was charged slowly over 30 minutes. The reaction was quenched by addition of sodium bisulfite (6.0 g) in water (60 mL). Acetic acid (23 mL) was charged over a period of 1 hour to adjust the pH of the reaction mixture between 6 and 7. The product was collected by filtration, washed with water and acetonitrile, and dried to give 7b (53 g, 98%). MS 286 [M+1].

### Example 8

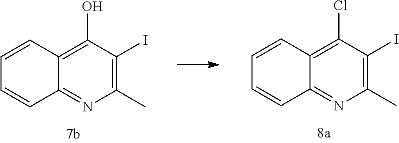

4-Hydroxy-3-iodo-2-methylquinoline 7b (25 g, 0.09 mol) was charged to a 1-L reactor. Ethyl acetate (250 mL) was charged, followed by triethylamine (2.45 mL, 0.02 mol) and phosphorus oxychloride (12 mL, 0.13 mol). The reaction mixture was heated to reflux until complete conversion (˜1 hour), then the mixture was cooled to 22° C. A solution of sodium carbonate (31.6 g, 0.3 mol) in water (500 mL) was charged. The mixture was stirred for 20 minutes. The aqueous layer was extracted with ethyl acetate (120 mL). The organic layers were combined and concentrated under vacuum to dryness. Acetone (50 mL) was charged. The solution was heated to 60° C. Water (100 mL) was charged, and the mixture was cooled to 22° C. The product was collected by filtration and dried to give 8a (25 g, 97.3% pure, 91.4% yield). MS 304 [M+1].

(Note: 8a is a known compound with CAS #1033931-93-9. See references: (a) J. Org Chem. 2008, 73, 4644-4649. (b) Molecules 2010, 15, 3171-3178. (c) Indian J. Chem. Sec B: Org. Chem. Including Med Chem. 2009, 488(5), 692-696.)

### Example 9

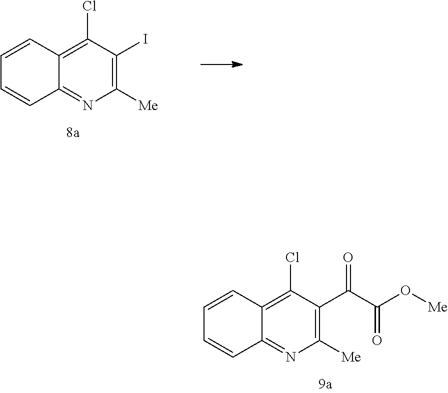 [SMILES: Cc1nc2ccccc2c(Cl)c1I ; COC(=O)C(=O)c1c(C)nc2ccccc2c1Cl]

8a (100 g, 0.33 mol) was charged to the reactor, followed by copper (I) bromide dimethyl sulfide complex (3.4 g, 0.017 mol) and dry THF (450 mL). The batch was cooled to −15 to −12° C. i-PrMgCl (2.0 M in THF, 173 mL, 0.346 mol) was charged into the reactor at the rate which maintained the batch temperature <−10° C. In a 2nd reactor, methyl chlorooxoacetate (33 mL, 0.36 mol) and dry THF (150 mL) were charged. The solution was cooled to −15 to −10° C. The content of the 1st reactor (Grignard/cuprate) was charged into the 2nd reactor at the rate which maintained the batch temperature <−10° C. The batch was agitated for 30 minutes at −10° C. Aqueous ammonium chloride solution (10%, 300 mL) was charged. The batch was agitated at 20-25° C. for 20 minutes and allowed to settle for 20 minutes. The aqueous layer was separated. Aqueous ammonium chloride solution (10%, 90 mL) and sodium carbonate solution (10%, 135 mL) were charged to the reactor. The batch was agitated at 20-25° C. for 20 minutes and allowed to settle for 20 minutes. The aqueous layer was separated. Brine (10%, 240 mL) was charged to the reactor. The batch was agitated at 20-25° C. for 20 minutes. The aqueous layer was separated. The batch was concentrated under vacuum to ˜¼ of the volume (about 80 mL left). 2-Propanol was charged (300 mL). The batch was concentrated under vacuum to ˜⅓ of the volume (about 140 mL left), and heated to 50° C. Water (70 mL) was charged. The batch was cooled to 20-25° C., stirred for 2 hours, cooled to −10° C. and stirred for another 2 hours. The solid was collected by filtration, washed with cold 2-propanol and water to provide 58.9 g of 9a obtained after drying (67.8% yield). ¹H NMR (400 MHz, CDCl₃): b 8.08 (d, 1H, J=12 Hz), 7.97 (d, 1H. J=12 Hz), 7.13 (t, 1H, J=8 Hz), 7.55 (t, 1H, J=8 Hz), 3.92 (s, 3H), 2.63 (s, 3H). ¹³C NMR (100 MHz, CDCl₃): δ 186.6, 161.1, 155.3, 148.2, 140.9, 132.0, 129.0, 128.8, 127.8, 123.8, 123.7, 53.7, 23.6.

### Example 10

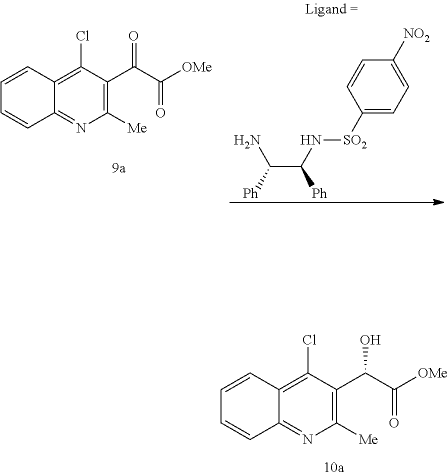 [SMILES: COC(=O)C(=O)c1c(C)nc2ccccc2c1Cl ; N[C@@H](c1ccccc1)[C@@H](NS(=O)(=O)c1ccc([N+](=O)[O-])cc1)c1ccccc1 ; COC(=O)[C@@H](O)c1c(C)nc2ccccc2c1Cl]

### Catalyst Preparation:

To a suitable sized, clean and dry reactor was charged dichloro(pentamethylcyclopentadienyl)rhodium (III) dimer (800 ppm relative to 9a, 188.5 mg) and the ligand (2000 ppm relative to 9a, 306.1 mg). The system was purged with nitrogen and then 3 mL of acetonitrile and 0.3 mL of triethylamine was charged to the system. The resulting solution was agitated at room temperature for not less than 45 minutes and not more than 6 hours.

### Reaction:

To a suitable sized, clean and dry reactor was charged 9a (1.00 equiv, 100.0 g (99.5 wt %), 377.4 mmol). The reaction was purged with nitrogen. To the reactor was charged acetonitrile (ACS grade, 4 L/Kg of 9a, 400 mL) and triethylamine (2.50 equiv, 132.8 mL, 943 mmol). Agitation was initiated. The 9a solution was cooled to Tᵢₙₜ=−5 to 0° C. and then formic acid (3.00 equiv, 45.2 mL, 1132 mmol) was charged to the solution at a rate to maintain T, not more than 20° C. The batch temperature was then adjusted to Tᵢₙₜ=−5 to −0° C. Nitrogen was bubbled through the batch through a porous gas dispersion unit (Wilmad-LabGlass No. LG-8680-110, VWR catalog number 14202-962) until a fine stream of bubbles was obtained. To the stirring solution at Tᵢₙₜ=−5 to 0° C. was charged the prepared catalyst solution from the catalyst preparation above. The solution was agitated at Tᵢₙₜ=−5 to 0° C. with the bubbling of nitrogen through the batch until HPLC analysis of the batch indicated no less than 98 A % conversion (as recorded at 220 nm, 10-14 h). To the reactor was charged isopropylacetate (6.7 L/Kg of 9a, 670 mL). The batch temperature was adjusted to Tᵢₙₜ=18 to 23° C. To the solution was charged water (10 L/Kg of 9a, 1000 mL) and the batch was agitated at Tᵢₙₜ=18 to 23° C. for no less than 20 minutes. The agitation was decreased and or stopped and the layers were allowed to separate. The lighter colored aqueous layer was cut. To the solution was charged water (7.5 L/Kg of 9a, 750 mL) and the batch was agitated at Tᵢₙₜ=18 to 23° C. for no less than 20 minutes. The agitation was decreased and or stopped and the layers were allowed to separate. The lighter colored aqueous layer was cut. The batch was then reduced to 300 mL (3 L/Kg of 9a) via distillation while maintaining Tₑₓₜ no more than 65° C. The batch was cooled to Tᵢₙₜ=35 to 45° C. and the batch was seeded (10 mg). To the batch at Tᵢₙₜ=35 to 45° C. was charged heptane (16.7 L/Kg of 9a, 1670 mL) over no less than 1.5 hours. The batch temperature was adjusted to Tᵢₙₜ=−2 to 3° C. over no less than 1 hour, and the batch was agitated at Tᵢₙₜ=−2 to 3° C. for no less than 1 hour. The solids were collected by filtration. The filtrate was used to rinse the reactor (Filtrate is cooled to Tᵢₙₜ=−2 to 3° C. before filtration) and the solids were suction dried for no less than 2 hours. The solids were dried until the LOD is no more than 4% to obtain 82.7 g of 10a (99.6-100 wt %, 98.5% ee, 82.5% yield). ¹H-NMR (CDCl₃, 400 MHz) δ: 8.20 (d, J=8.4 Hz, 1H), 8.01 (d, J=8.4 Hz, 1H), 7.73 (t, J=7.4 Hz, 1H), 7.59 (t, J=7.7 Hz, 1H), 6.03 (s, 1H), 3.93 (s, 1H), 3.79 (s, 3H), 2.77 (s, 3H). ¹³C-NMR (CDCl₃, 100 MHz) δ: 173.5, 158.3, 147.5, 142.9, 130.7, 128.8, 127.7, 127.1, 125.1, 124.6, 69.2, 53.4, 24.0.

### Example 11

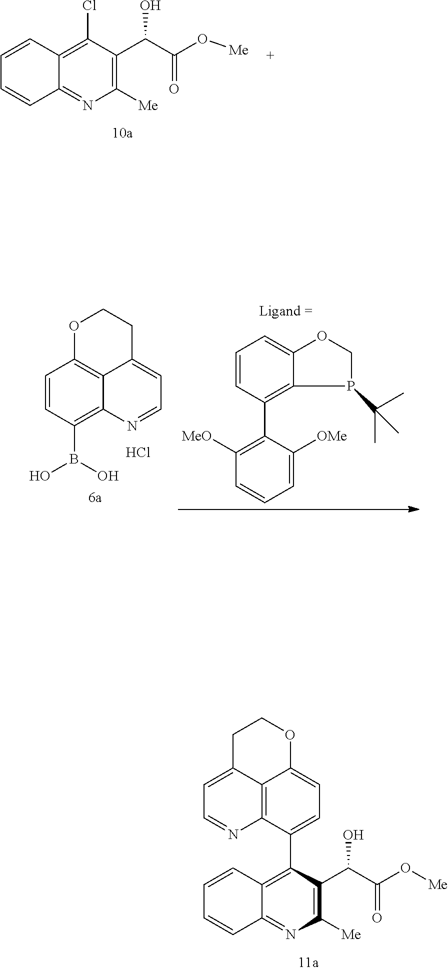

10a (2.45 kg, 96.8% purity, 8.9 mol), 6a (2.5 kg, 88.7% purity, 8.82 mol), tris(dibenzylideneacetone)dipalladium(0) (Pd₂dba₃, 40 g, 0.044 mol), (S)-3-tert-butyl-4-(2,6-dimethoxyphenyl)-2,3-dihydrobenzo[d][1,3]oxaphosphole (32 g, 0.011 mol), sodium carbonate (1.12 kg, 10.58 mol), 1-pentanol (16.69 L), and water (8.35 L) were charged to the reactor. The mixture was de-gassed by sparging with argon for 10-15 minutes, was heated to 60-63° C., and was agitated until HPLC analysis of the reaction shows <1 A % (220 nm) of the 6a relative to the combined two atropisomer products (˜15 hours). The batch was cooled to 18-23° C. Water (5 L) and heptane (21 L) were charged. The slurry was agitated for 3-5 hours. The solids were collected by filtration, washed with water (4 L) and heptane/toluene mixed solvent (2.5 L toluene/5 L heptane), and dried. The solids were dissolved in methanol (25 L) and the resulting solution was heated to 50° C. and circulated through a CUNO carbon stack filter. The solution was distilled under vacuum to ˜5 L. Toluene (12 L) was charged. The mixture was distilled under vacuum to ˜5 L and cooled to 22° C. Heptane (13 L) was charged to the contents over 1 hour and the resulting slurry was agitated at 20-25° C. for 3-4 hours. The solids were collected by filtration and washed with heptanes to provide 2.58 kg of 11a obtained after drying (73% yield). ¹H NMR (400 MHz, CDCl₃): δ 8.63 (d, 1H, J=8 Hz), 8.03 (d, 1H, J=12 Hz), 7.56 (t, 1H, J=8 Hz), 7.41 (d, 1H, J=8 Hz), 7.19 (t, 1H, J=8 Hz), 7.09 (m, 2H), 7.04 (d, 1H, J=8 Hz), 5.38 (d, 1H, J=8 Hz), 5.14 (d, 1H, J=8 Hz), 4.50 (1, 2H, J=4 Hz), 3.40 (s, 3H), 3.25 (t, 2H, J=4 Hz), 2.91 (s, 3H). ¹³C NMR (100 MHz, CDCl₃): δ 173.6, 158.2, 154.0, 150.9, 147.3, 147.2, 145.7, 141.3, 132.9, 123.0, 129.4, 128.6, 127.8, 126.7, 126.4, 125.8, 118.1, 117.3, 109.9, 70.3, 65.8, 52.3, 28.5, 24.0.

### Example 12

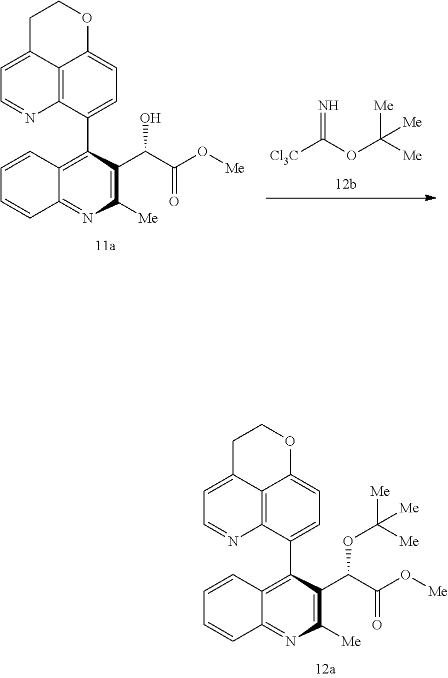

To a suitable clean and dry reactor under a nitrogen atmosphere was charged 11a (5.47 Kg, 93.4 wt %, 1.00 equiv, 12.8 mol) and fluorobenzene (10 vols, 51.1 kg) following by trifluoromethanesulfonimide (4 mol %, 143 g, 0.51 mol) as a 0.5 M solution in DCM (1.0 Kg). The batch temperature was adjusted to 35-41° C. and agitated to form a fine slurry. To the mixture was slowly charged t-butyl-2,2,2-trichloroacetimidate 12b as a 50 wt % solution (26.0 Kg of t-butyl-2,2,2-trichloroacetimidate (119.0 mol, 9.3 equiv), the reagent was −48-51 wt % with the remainder 52-49 wt % of the solution being ˜1.8:1 wt:wt heptane:fluorobenzene) over no less than 4 hours at Tᵢₙₜ=35-41° C. The batch was agitated at Tᵢₙₜ=35-41° C. until HPLC conversion (308 nm) was >96 A %, then cooled to Tᵢₙₜ=20-25° C. and then triethylamine (0.14 equiv, 181 g, 1.79 mol) was charged followed by heptane (12.9 Kg) over no less than 30 minutes. The batch was agitated at Tᵢₙₜ=20-25° C. for no less than 1 hour. The solids were collected by filtration. The reactor was rinsed with the filtrate to collect all solids. The collected solids in the filter were rinsed with heptane (11.7 Kg). The solids were charged into the reactor along with 54.1 Kg of DMAc and the batch temperature adjusted to Tᵢₙₜ=70-75° C. Water (11.2 Kg) was charged over no less than 30 minutes while the batch temperature was maintained at Tᵢₙₜ=65-75° C. 12a seed crystals (34 g) in water (680 g) was charged to the batch at Tᵢₙₜ=65-75° C. Additional water (46.0 Kg) was charged over no less than 2 hours while maintaining the batch temperature at Tᵢₙₜ=65-75° C. The batch temperature was adjusted to Tᵢₙₜ=18-25° C. over no less than 2 hours and agitated for no less than 1 hour. The solids were collected by filtration and the filtrate used to rinse the reactor. The solids were washed with water (30 Kg) and dried under vacuum at no more than 45° C. until the LOD<4% to obtain 12a (5.275 Kg, 99.9 A % at 220 nm, 99.9 wt % via HPLC wt % assay, 90.5% yield). ¹H-NMR (CDCl₃, 400 MHz) δ: 8.66-8.65 (m, 1H), 8.05 (d, J=8.3 Hz, 1H), 7.59 (t, J=7.3 Hz, 1H), 7.45 (d, J=7.8 Hz, 1H), 7.21 (t. J=7.6 Hz, 1H), 7.13-7.08 (m, 3H), 5.05 (s, 1H), 4.63-4.52 (m, 2H), 3.49 (s, 3H), 3.41-3.27 (m, 2H), 3.00 (s, 3H), 0.97 (s, 9H). ¹³C-NMR (CDCl₃, 100 MHz) δ: 172.1, 159.5, 153.5, 150.2, 147.4, 146.9, 145.4, 140.2, 131.1, 130.1, 128.9, 128.6, 128.0, 127.3, 126.7, 125.4, 117.7, 117.2, 109.4, 76.1, 71.6, 65.8, 51.9, 28.6, 28.0, 25.4.

### Example 13

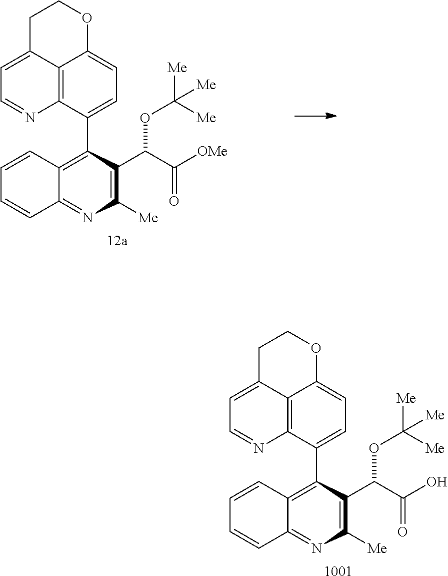

To a suitable clean and dry reactor under a nitrogen atmosphere was charged 12a (9.69 Kg, 21.2 mol) and ethanol (23.0 Kg). The mixture was agitated and the batch temperature was maintained at Tᵢₙₜ=20 to 25=C. 2 M sodium hydroxide (17.2 Kg) was charged at Tᵢₙₜ=20 to 25° C. and the batch temperature was adjusted to Tᵢₙₜ=60-65° C. over no less than 30 minutes. The batch was agitated at Tᵢₙₜ=60-65° C. for 2-3 hours until HPLC conversion was >99.5% area (12a is <0.5 area %). The batch temperature was adjusted to Tᵢₙₜ=50 to 55° C. and 2M aqueous HCl (14.54 Kg) was charged. The pH of the batch was adjusted to pH 5.0 to 5.5 (target pH 5.2 to 5.3) via the slow charge of 2M aqueous HCl (0.46 Kg) at Tᵢₙₜ=50 to 55° C. Acetonitrile was charged to the batch (4.46 Kg) at Tᵢₙₜ=50 to 55° C. A slurry of seed crystals (1001, 20 g in 155 g of acetonitrile) was charged to the batch at Tᵢₙₜ=50 to 55° C. The batch was agitated at Tᵢₙₜ=50 to 55° C. for no less than 1 hour (1-2 hours). The contents were vacuum distilled to ˜3.4 vol (32 L) while maintaining the internal temperature at 45-55° C. A sample of the batch was removed and the ethanol content was determined by GC analysis; the criterion was no more than 10 wt % ethanol. If the ethanol wt % was over 10%, an additional 10% of the original volume was distilled and sampled for ethanol wt %. The batch temperature was adjusted to Tᵢₙₜ=18-22° C. over no less than 1 hour. The pH of the batch was verified to be pH=5-5.5 and the pH was adjusted, if necessary, with the slow addition of 2 M HCl or 2 M NaOH aqueous solutions. The batch was agitated at Tᵢₙₜ=18-22° C. for no less than 6 hours and the solids were collected by filtration. The filtrate/mother liquid was used to remove all solids from reactor. The cake with was washed with water (19.4 Kg) (water temperature was no more than 20° C.). The cake was dried under vacuum at no more than 60° C. for 12 hours or until the LOD was no more than 4% to obtain 1001 (9.52 Kg, 99.6 A % 220 nm, 97.6 wt % as determined by HPLC wt % assay, 99.0% yield).

### Example 14

### Preparation of 12b

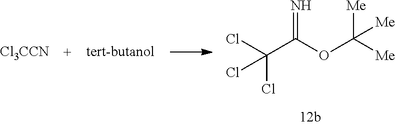 [SMILES: "Cl3CCN" ; "tert-butanol" ; CC(C)(C)OC(=N)C(Cl)(Cl)Cl]

To a 2 L 3-neck dried reactor under a nitrogen atmosphere was charged 3 mol % (10.2 g, 103 mmol) of sodium tert-butoxide and 1.0 equivalent of tert-butanol (330.5 mL, 3.42 mol). The batch was heated at Tᵢₙₜ=50 to 60° C. until most of the solid was dissolved (˜1 to 2 h). Fluorobenzene (300 mL) was charged to the batch. The batch was cooled to Tᵢₙₜ=<−5° C. (−10 to −5° C.) and 1.0 equivalent of trichloroacetonitrile (350 mL, 3.42 mol) was charged to the batch. The addition was exothermic so the addition was controlled to maintain Tᵢₙₜ=<−5° C. The batch temperature was increased to Tᵢₙₜ=15 to 20° C. and heptane (700 mL) was charged. The batch was agitated at Tᵢₙₜ=15 to 20° C. for no less than 1 h. The batch was passed through a short Celite (Celite 545) plug to produce 1.256 Kg of 12b. Proton NMR with the internal standard indicated 54.6 wt % 12b, 27.8 wt % heptane and 16.1 wt % fluorobenzene (overall yield: 92%).

Compounds 1002-1055 are prepared analogously to the procedure described in Examples 11, 12 and 13 using the appropriate boronic acid or boronate ester. The synthesis of said boronic acid or boronate ester fragments are described in WO 2007/131350 and WO 2009/062285, both of which are herein incorporated by reference.

### Table of Compounds

The following table lists compounds representative of the invention. All of the compounds in Table 1 are synthesized analogously to the Examples described above. It will be apparent to a skilled person that the analogous synthetic routes may be used, with appropriate modifications, to prepare the compounds of the invention as described herein.

Retention times (t_{R}) for each compound are measured using the standard analytical HPLC conditions described in the Examples. As is well known to one skilled in the art, retention time values are sensitive to the specific measurement conditions. Therefore, even if identical conditions of solvent, flow rate, linear gradient, and the like are used, the retention time values may vary when measured, for example, on different HPLC instruments. Even when measured on the same instrument, the values may vary when measured, for example, using different individual HPLC columns, or, when measured on the same instrument and the same individual column, the values may vary, for example, between individual measurements taken on different occasions.

TABLE 1

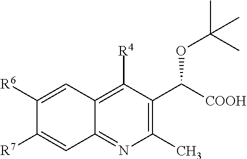

| | | | | | MS |
|---|---|---|---|---|---|
| | | | | t_{R} | (M + |
| Cpd | R⁴ | R⁶ | R⁷ | (min) | H)⁺ |
| 1001 | 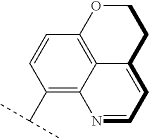 | H | H | 3.7 | 443.2 |
| 1002 | 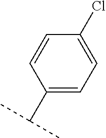 | CH₃ | H | 4.7 | 398.1/ 400.1 |
| 1003 | 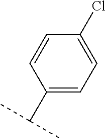 | H | CH₃ | 4.6 | 398.1/ 400.1 |
| 1004 | 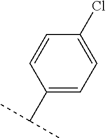 | H | F | 4.5 | 402.2/ 404.1 |
| 1005 | 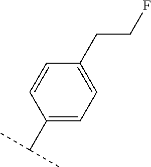 | H | H | 3.9 | 396.2 |
| 1006 | 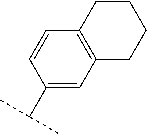 | H | H | 5.1 | 404.2 |
| 1007 | 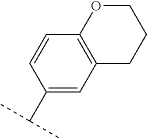 | H | H | 4.3 | 406.2 |
| 1008 | 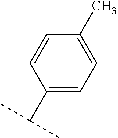 | H | H | 4.5 | 364.2 |
| 1009 | 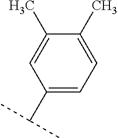 | H | H | 4.8 | 378.2 |
| 1010 | 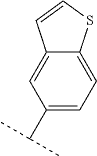 | H | H | 4.7 | 406.2 |
| 1011 | 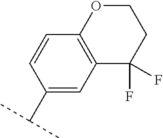 | H | H | 3.9 | 442.1 |
| 1012 | 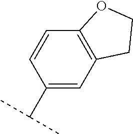 | H | H | 3.7 | 392.1 |
| 1013 | 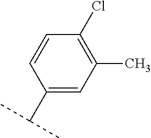 | H | H | 5.0 | 398.1/ 400.1 |
| 1014 | 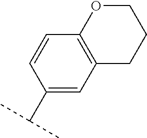 | H | CH₃ | 4.3 | 420.1 |
| 1015 | 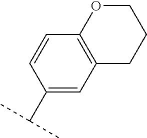 | F | H | 4.9 | 424.2 |
| 1016 | 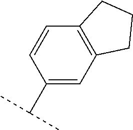 | H | H | 4.4 | 390.1 |
| 1017 | 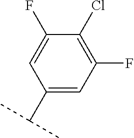 | H | H | 5.2 | 420.1/ 422.1 |
| 1018 | 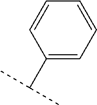 | H | CH₃ | 4.4 | 364.2 |
| 1019 | 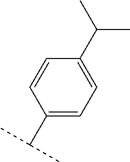 | H | CH₃ | 5.5 | 406.2 |
| 1020 | 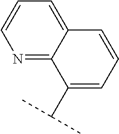 | H | CH₃ | 3.6 | 415.2 |
| 1021 | 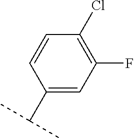 | H | CH₃ | 4.4 | 416.1/ 418.2 |
| 1022 | 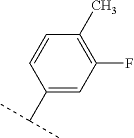 | H | CH₃ | 4.8 | 396.2 |
| 1023 | 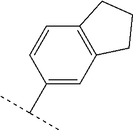 | H | CH₃ | 4.6 | 404.2 |
| 1024 | 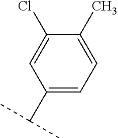 | H | H | 4.9 | 398.1/ 400.1 |
| 1025 | 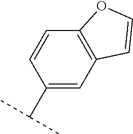 | H | H | 3.9 | 390.1 |
| 1026 | 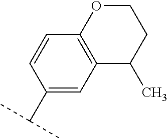 | H | H | 4.1 | 420.2 |
| 1027 | 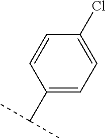 | CH₂CH₃ | H | 5.5 | 412.2/ 414.2 |
| 1028 | 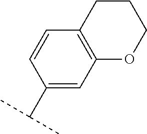 | H | H | 3.7 | 406.2 |
| 1029 | 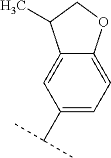 | H | H | 4.6 | 406.2 |
| 1030 | 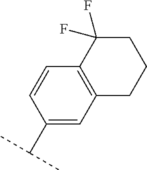 | H | H | 4.1 | 440.2 |
| 1031 | 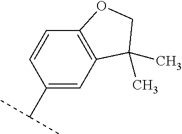 | H | H | 4.9 | 420.2 |
| 1032 | 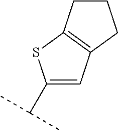 | H | H | 5.0 | 396.2 |
| 1033 | 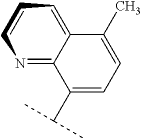 | H | H | 3.6 | 415.3 |
| 1034 | 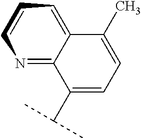 | H | CH₃ | 3.9 | 429.2 |
| 1035 | 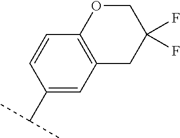 | H | H | 5.2 | 442.2 |
| 1036 | 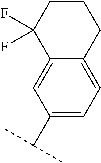 | H | H | 5.4 | 440.1 |
| 1037 | 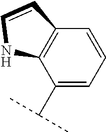 | H | H | 4.6 | 398.2 |
| 1038 | 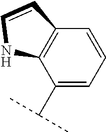 | H | CH₃ | 4.9 | 403.2 |
| 1039 | 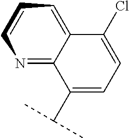 | H | CH₃ | 4.5 | 449.2/ 451.2 |
| 1040 | 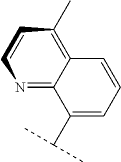 | H | CH₃ | 3.4 | 429.3 |
| 1041 | 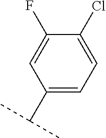 | H | H | 4.5 | 402.1/ 404.1 |
| 1042 | 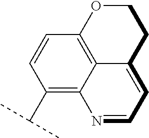 | H | —CH₃ | 3.6 | 457.3 |
| 1043 | 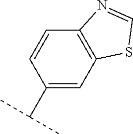 | H | H | 3.0 | 407.1 |
| 1044 | 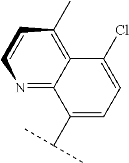 | H | Me | 5.0 | 463.2/ 465.2 |
| 1045 | 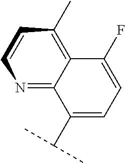 | H | Me | 4.4 | 447.3 |
| 1046 | 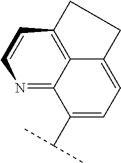 | H | Me | 3.1 | 441.2 |
| 1047 | 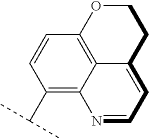 | H | Cl | 3.1 | 447.2/ 479.2 |
| 1048 | 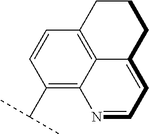 | H | H | 3.2 | 441.3 |
| 1049 | 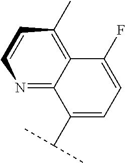 | H | H | 4.1 | 433.3 |
| 1050 | 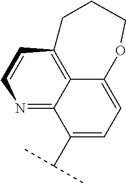 | H | H | 3.8 | 457.2 |
| 1051 | 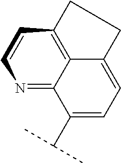 | H | H | 2.8 | 472.2 |
| 1052 | 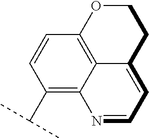 | Me | H | 3.7 | 457.2 |
| 1053 | 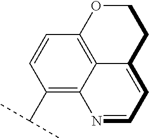 | Cl | H | 3.0 | 477.3/ 479.3 |
| 1054 | 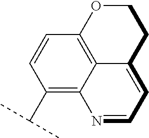 | F | H | 2.8 | 461.3 |
| 1055 | 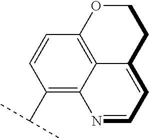 | F | Me | 2.9 | 475.1 |

Each of the references including all patents, patent applications and publications cited in the present application is incorporated herein by reference in its entirety, as if each of them is individually incorporated. Further, it would be appreciated that, in the above teaching of invention, the skilled in the art could make certain changes or modifications to the invention, and these equivalents would still be within the scope of the invention defined by the appended claims of the application.

1. A process to prepare Compound 1001 or a salt thereof: 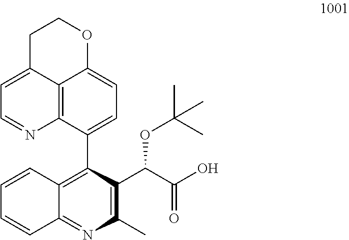
according to the following General Scheme IA: 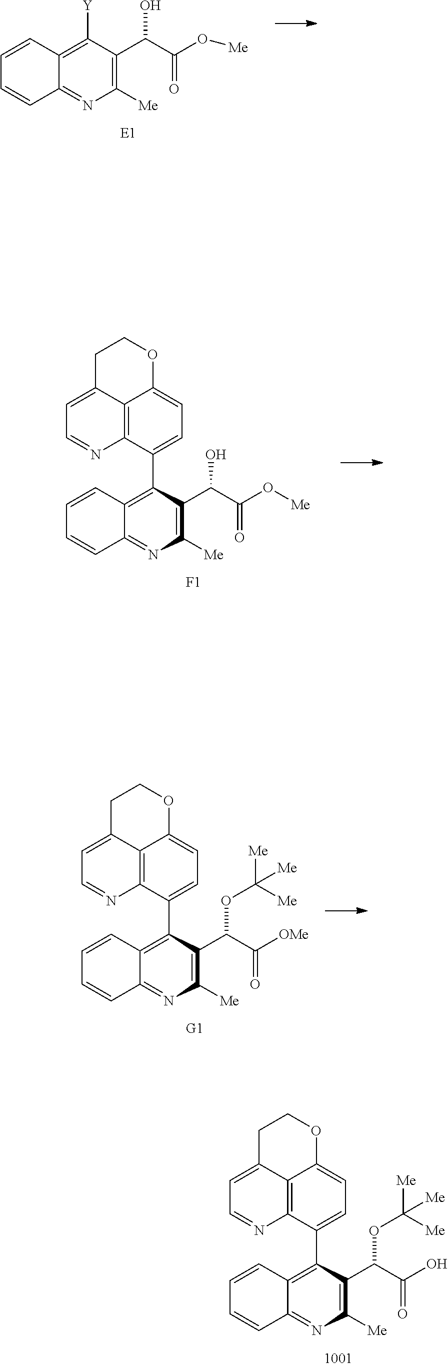
wherein Y is I, Br or Cl;
wherein the process comprises:
coupling aryl halide E1 under diastereoselective Suzuki coupling conditions in the presence of a chiral biaryl monophosphorus ligand having Formula (AA): 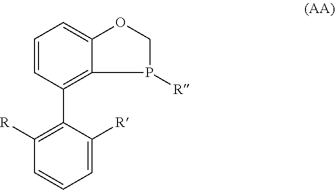
wherein R═R′═H; R″=tert-butyl; or R═OMe; R′═H; R″=tert-butyl; or R═N(Me)₂; R′═H; R″=tert-butyl;
in combination with a palladium catalyst or precatalyst, and a base and a boronic acid or boronate ester in a solvent mixture;
converting chiral alcohol F1 to tert-butyl ether G1 under BrØnstead- or Lewis-acid catalysis with a source tert-butyl cation or its equivalent;
saponifying ester G1 to Compound 1001 in a solvent mixture; and
optionally converting Compound 1001 to a salt.

2. The process according to claim 1, wherein the palladium catalyst or precatalyst is tris(dibenzylideneacetone)dipalladium(0) and the chiral biaryl monophosphorus ligand is ligand Q:

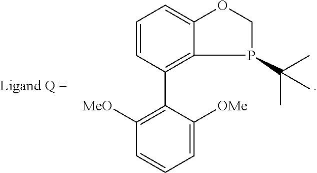 [SMILES: COc1cccc(OC)c1-c1cccc2c1[P@](C(C)(C)C)CO2]

3. The process according to claim 1, wherein the boronic acid or boronate ester is a boronic acid selected from:

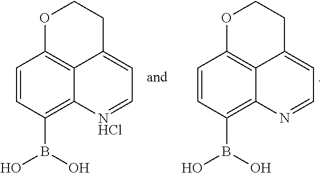

4. The process according to claim 1, wherein the boronic acid is prepared according to the following General Scheme III: 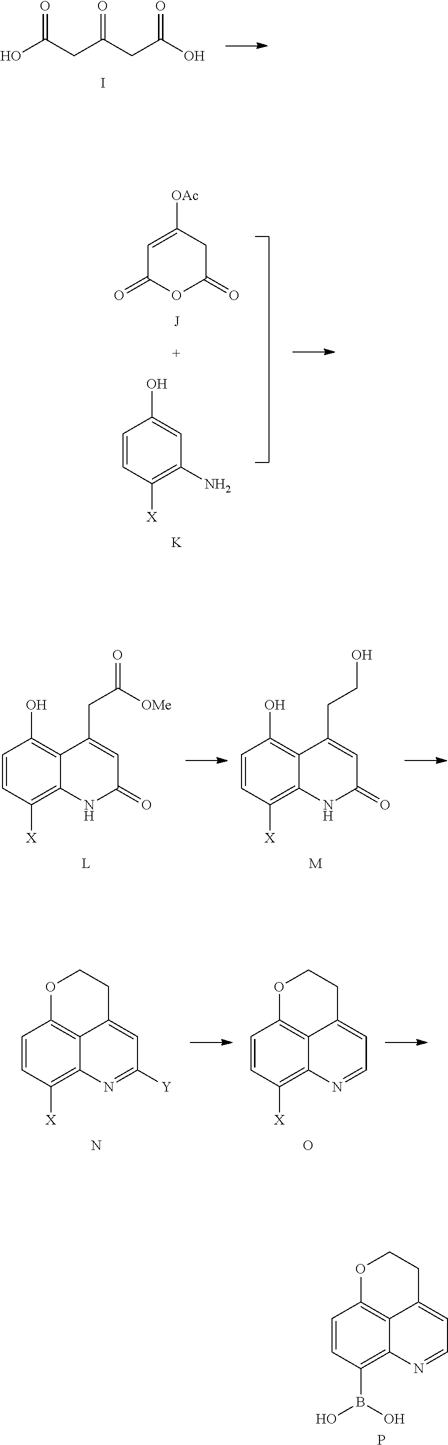
wherein:
X is Br or I;
Y is Br or Cl; and
R₁ and R₂ may either be absent or linked to form a cycle;
wherein the process comprises:
converting diacid I to cyclic anhydride J;
condensing anhydride J with meta-aminophenol K to give quinolone L;
reducing the ester of compound L to give alcohol M;
cyclizing alcohol M to give tricyclic quinoline N by activating the alcohol as its corresponding alkyl chloride or alkyl bromide;
reductively removing halide Y under acidic conditions in the presence of a reductant to give compound O;
converting halide X in compound O to the corresponding boronic acid P, sequentially via the corresponding intermediate aryl lithium reagent and boronate ester; and
optionally converting Compound P to a salt thereof.

5. The process according to claim 1, wherein the chiral alcohol F1 is converted to tert-butyl ether G1 using trifluoromethanesulfonimide as the catalyst and t-butyl-trichloroacetimidate as source tert-butyl cation.

6. A process to prepare Compound 1001 or salt thereof 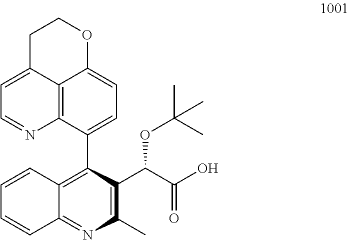
according to the following General Scheme IIA: 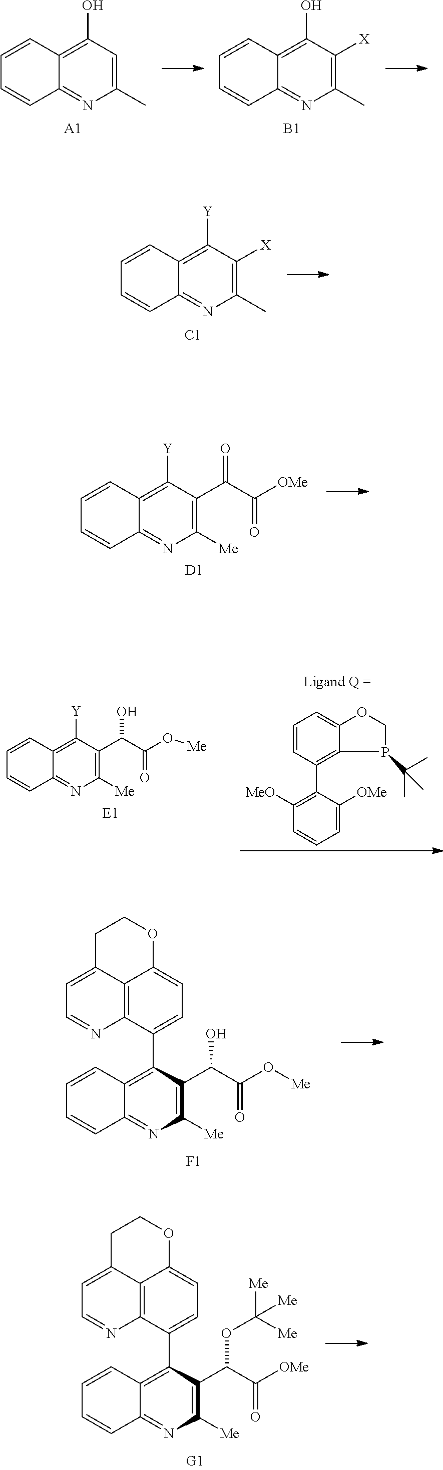 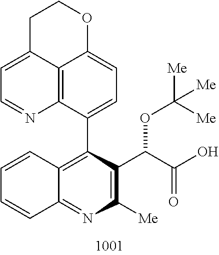
wherein:
X is I or Br; and
Y is Cl when X is Br or I, or Y is Br when X is I, or Y is I;
wherein the process comprises:
converting 4-hydroxyquinoline A1 to phenol B1 via a regioselective halogenation reaction at the 3-position of the quinoline core;
converting phenol B1 to aryl dihalide C1 through activation of the phenol with an activating reagent and subsequent treatment with a halide source in the presence of an organic base;
converting aryl dihalide C1 to ketone D1 by chemoselectively transforming the 3-halo group to an aryl metal reagent and then reacting the aryl metal reagent with an activated carboxylic acid;
stereoselectively reducing ketone D1 to chiral alcohol E1 by asymmetric ketone reduction methods;
diastereoselectively coupling aryl halide E1 under Suzuki coupling reaction conditions in the presence of a chiral phosphine ligand Q in combination with a palladium catalyst or precatalyst, a base and a boronic acid or boronate ester in a solvent mixture;
converting chiral alcohol F1 to tert-butyl ether G1 under BrØnstead- or Lewis-acid catalysis with a source tert-butyl cation or its equivalent;
saponifying ester G1 to Compound 1001 in a solvent mixture; and
optionally converting Compound 1001 to a salt thereof.

7. The process according to claim 6, wherein the palladium catalyst or precatalyst is tris(dibenzylideneacetone)dipalladium(0).

8. The process according to claim 6, wherein the boronic acid or boronate ester is a boronic acid selected from:

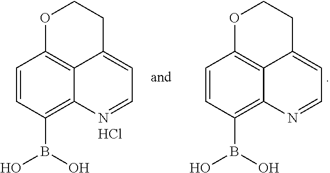 [SMILES: OB(O)c1ccc2c3c(ccnc13)CCO2 ; Cl ; OB(O)c1ccc2c3c(ccnc13)CCO2]

9. The process according to claim 6, wherein the boronic acid is prepared according to the following General Scheme III: 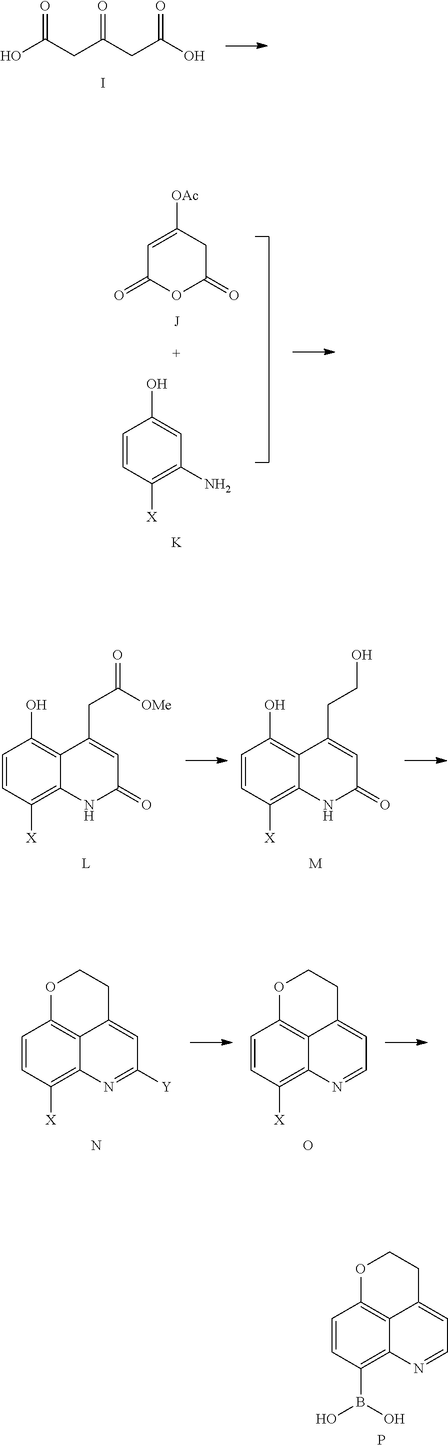
wherein:
X is Br or I;
Y is Br or Cl; and
R₁ and R₂ may either be absent or linked to form a cycle;
wherein the process comprises:
converting diacid I to cyclic anhydride J;
condensing anhydride J with meta-aminophenol K to give quinolone L;
reducing the ester of compound L to give alcohol M
cyclizing alcohol M to give tricyclic quinoline N via activation of the alcohol as its corresponding alkyl chloride or alkyl bromide;
deductively removing halide Y under acidic conditions with a reductant to give compound O;
converting halide X in compound O to the corresponding boronic acid P, sequentially via the corresponding intermediate aryl lithium reagent and boronate ester; and
optionally converting compound P to a salt thereof.

10. A process according to claim 6, wherein ketone D1 is stereoselectively reduced to chiral alcohol E1 with ligand Z, 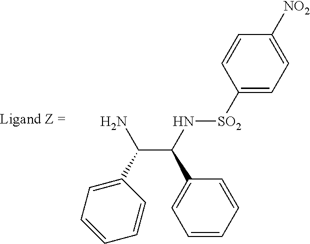 [SMILES: N[C@@H](c1ccccc1)[C@@H](NS(=O)(=O)c1ccc([N+](=O)[O-])cc1)c1ccccc1]
dichloro(pentamethylcyclopentadienyl)rhodium (III) dimer and formic acid.

11. The process according to claim 6, wherein the chiral alcohol F1 is converted to tert-butyl ether G1 with trifluoromethanesulfonimide as the catalyst and t-butyl-trichloroacetimidate.

12. A process to prepare a compound of Formula (I) or a salt thereof: 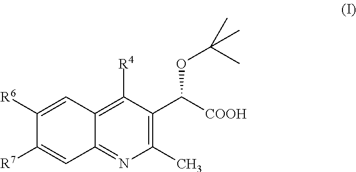
wherein:
R⁴ is selected from the group consisting of: 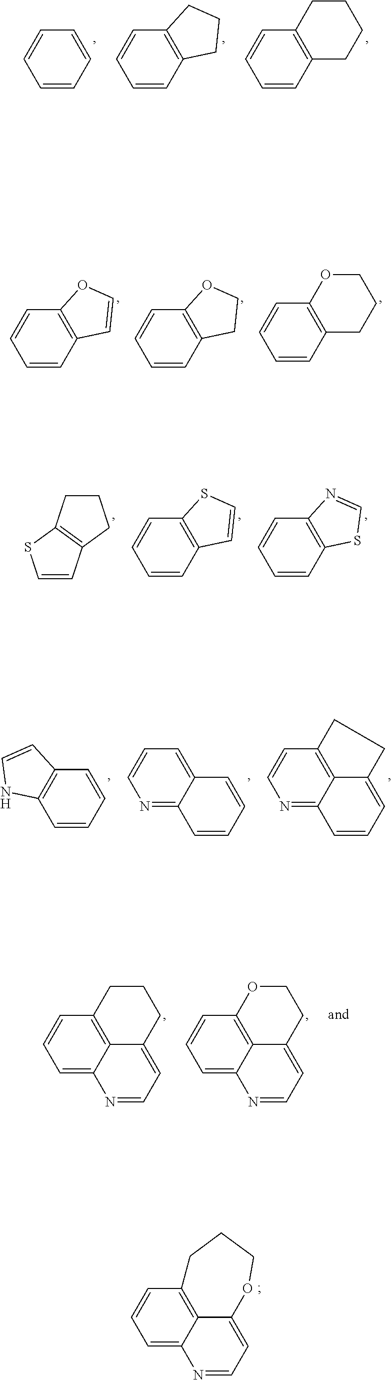
and
R⁶ and R⁷ are each independently selected from H, halo and (C₁₋₆)alkyl;
according to the following General Scheme I: 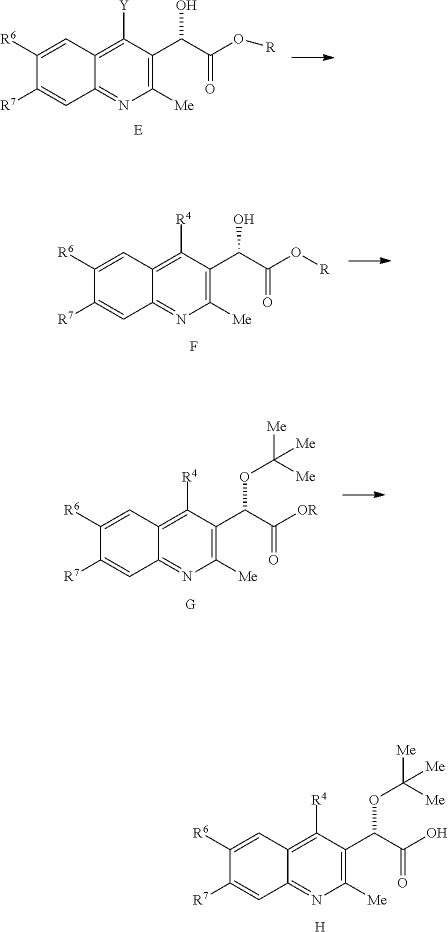
wherein:
Y is I, Br or Cl; and
R is (C₁₋₆)alkyl;
wherein the process comprises:
coupling aryl halide E under diastereoselective Suzuki coupling conditions in the presence of a chiral biaryl monophosphorus ligand having Formula (AA): 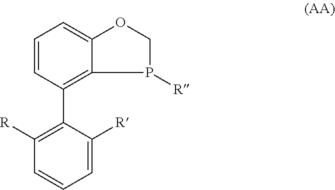
wherein R═R′═H; R″=tert-butyl; or R═OMe; R′═H; R″=tert-butyl; or R═N(Me)₂; R′═H; R″=tert-butyl;
in combination with a palladium catalyst or precatalyst, and a base and a boronic acid or boronate ester in a solvent mixture;
converting chiral alcohol F to tert-butyl ether G under BrØnstead- or Lewis-acid catalysis with a source tert-butyl cation or its equivalent;
saponifying ester G to inhibitor H in a solvent mixture; and
optionally converting inhibitor H to a salt.

13. The process according to claim 12, wherein the palladium catalyst or precatalyst is tris(dibenzylideneacetone)dipalladium(0) and the chiral biaryl monophosphorus ligand is ligand Q:

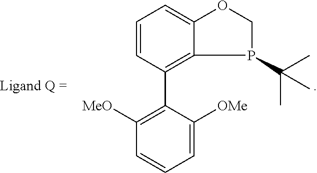 [SMILES: COc1cccc(OC)c1-c1cccc2c1[P@](C(C)(C)C)CO2]

14. The process according to claim 12, wherein the chiral alcohol F is converted to tert-butyl ether G with trifluoromethanesulfonimide as the catalyst and t-butyl-trichloroacetimidate.

15. A process to prepare a compound of Formula (I) or a salt thereof: 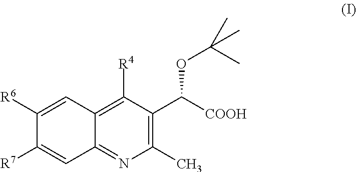
wherein:
R⁴ is selected from the group consisting of: 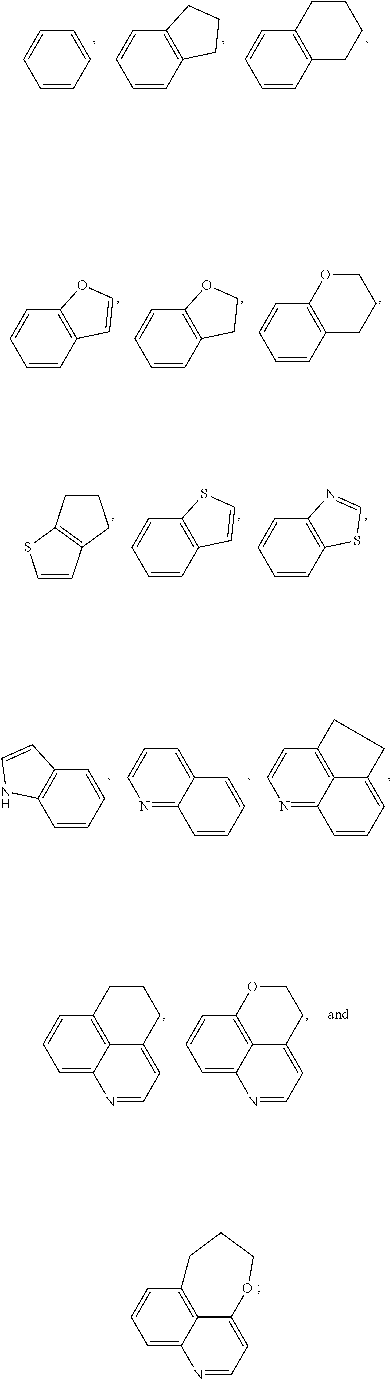
and
R⁶ and R⁷ are each independently selected from H, halo and (C₁₋₆)alkyl;
according to the following General Scheme II: 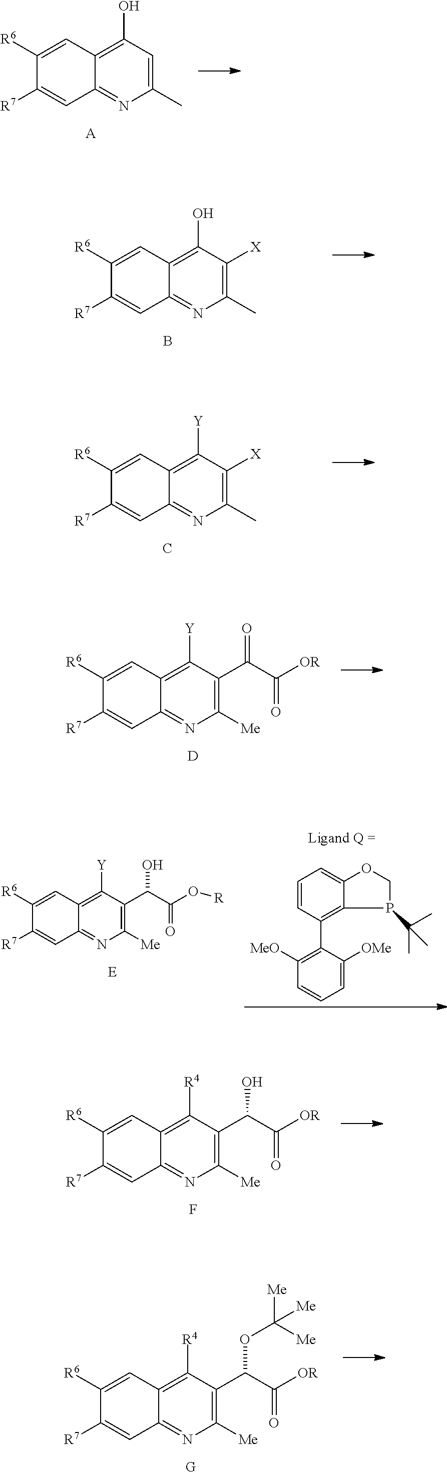 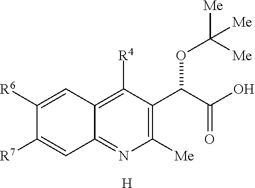
wherein:
X is I or Br;
Y is Cl when X is Br or I, or Y is Br when X is I, or Y is I; and
R is (C₁₋₆)alkyl;
wherein the process comprises:
converting 4-hydroxyquinoline A to phenol B via a regioselective halogenation reaction at the 3-position of the quinoline core;
converting phenol B to aryl dihalide C through activation of the phenol with an activating reagent and subsequent treatment with a halide source in the presence of an organic base;
converting aryl dihalide C to ketone D by chemoselectively transforming the 3-halo group to an aryl metal reagent and then reacting the aryl metal reagent with an activated carboxylic acid;
stereoselectively reducing ketone D to chiral alcohol E by asymmetric ketone reduction methods;
diastereoselectively coupling of aryl halide E with R⁴ in the presence of phosphine ligand Q in combination with a palladium catalyst or precatalyst, a base and a boronic acid or boronate ester in a solvent mixture;
converting chiral alcohol F to tert-butyl ether G under BrØnstead- or Lewis-acid catalysis with a source tert-butyl cation or its equivalent;
saponifying ester G to inhibitor H in a solvent mixture; and
optionally converting inhibitor H to a salt thereof.

16. The process according to claim 15, wherein the palladium catalyst or precatalyst is tris(dibenzylideneacetone)dipalladium(0).

17. A process according to claim 15, wherein ketone D is stereoselectively reduced to chiral alcohol E with ligand Z, 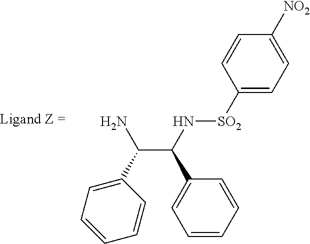 [SMILES: N[C@@H](c1ccccc1)[C@@H](NS(=O)(=O)c1ccc([N+](=O)[O-])cc1)c1ccccc1]
dichloro(pentamethylcyclopentadienyl)rhodium (III) dimer and formic acid.

18. The process according to claim 15, wherein the chiral alcohol F is converted to tert-butyl ether G with trifluoromethanesulfonimide as the catalyst and t-butyl-trichloroacetimidate.

19. The process according to claim 4, wherein the halide X in compound O is converted to the corresponding boronic acid P, in the presence of toluene.

20. The process according to claim 3, wherein the boronic acid or boronate ester is:

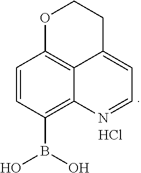 [SMILES: OB(O)c1ccc2c3c(ccnc13)CCO2 ; Cl]

21. The process according to claim 9, wherein the halide X in compound O is converted to the corresponding boronic acid P, in the presence of toluene.

22. The process according to claim 8, wherein the boronic acid or boronate ester is: 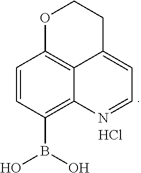 [SMILES: OB(O)c1ccc2c3c(ccnc13)CCO2 ; Cl]
